(19) United States
(12) Patent Application Publication
(10) Pub. No.: US 2025/0182877 A1
(43) Pub. Date: Jun. 5, 2025

(54) USER AUXILIARY INFORMATION OUTPUT DEVICE, USER AUXILIARY INFORMATION OUTPUT SYSTEM, AND USER AUXILIARY INFORMATION OUTPUT METHOD

(71) Applicant: Olympus Corporation, Tokyo (JP)

(72) Inventors: Kazuo KANDA, Tokyo (JP); Yoshiyuki FUKUYA, Sagamihara-shi (JP); Tomoko GOCHO, Tokyo (JP); Osamu NONAKA, Sagamihara-shi (JP)

(21) Appl. No.: 19/045,876

(22) Filed: Feb. 5, 2025

Related U.S. Application Data
(62) Division of application No. 17/592,403, filed on Feb. 3, 2022.

(30) Foreign Application Priority Data
Feb. 15, 2021 (JP) 2021-021827

(51) Int. Cl. G16H 20/60; G16H 50/30

(52) U.S. Cl. CPC G16H 20/60; G16H 50/30

(57) ABSTRACT

A user auxiliary information output device of the present invention comprises a user status information inputter for inputting status information of a user, and an advice outputter for outputting advice relating to user diet, using database information that is a combination of ingredients and cooking methods, obtained in accordance with the status information.

### CROSS-REFERENCE TO RELATED APPLICATIONS

The present application is a divisional application of pending U.S. patent application Ser. No. 17/592,403 (referred to as “the '403 application” and incorporated herein by reference), filed on Feb. 3, 2022, and titled “USER AUXILIARY INFORMATION OUTPUT DEVICE, USER AUXILIARY INFORMATION OUTPUT SYSTEM, AND USER AUXILIARY INFORMATION OUTPUT METHOD”. Benefit is claimed, under 35 U.S.C. § 119, to the filing date of prior Japanese Patent Application No. 2021-021827 (incorporated herein by reference) filed on Feb. 15, 2021. These applications are expressly incorporated herein by reference. The scope of the present invention is not limited to any requirements of the specific embodiments described in the applications.

### BACKGROUND OF THE INVENTION

### 1. Field of the Invention

The present invention relates to a user auxiliary information output device, user auxiliary information output system, and user auxiliary information output method that are capable of providing advice concerning proper diet in accordance with user conditions.

### 2. Description of the Related Art

A patient who has been admitted to a hospital takes meals inside the hospital in accordance with their condition. Although hospital meals will change in accordance with the conditions of a patient, it is necessary to share information among various people, such as patients, doctors and nurses, nutritionists, cooks, dining staff, etc. A hospital food management system that is capable of sharing information relating to food menus amongst these people has been disclosed in Japanese patent No 6442100 (hereafter referred to as “patent publication 1”).

With the hospital food management system disclosed in patent publication 1, people involved within the hospital share information relating to hospital food, and meals are provided in accordance with patient conditions. Specifically, even in cases where food menus have changed in accordance with change in patient conditions, or change in consultation planning, or changes in procedural planning, it is possible for patients, doctors, nurses, dining staff, and nutritionists to share information relating to meal changes.

However, with patent publication 1, after a patient has been discharged from hospital, it is necessary for the patient to take care of their own meals. Also, even if a user is not admitted to hospital, they must also take care of their meals themselves in accordance with their condition. Also, depending on the type of examination, it is often necessary to pay attention to meals eaten before an examination. However, since taking care of these meals is entrusted to the user, there may also be cases where appropriate dietary management is not performed.

Further, there may be cases where even if a user hopes to receive suitable meals in accordance with their own conditions, they might not be aware of shops and services that are capable of providing suitable meals. There are also cases where the user (or a set of more than one users (such as a husband and wife for example) does not know what food ingredients and/or cooking methods to use to obtain suitable meals in accordance with their own conditions. There are also cases where the user does not even know simply whether or not ingredients and food in front of them should be eaten.

### SUMMARY OF THE INVENTION

The present inventions provide a user auxiliary information output device, user auxiliary information output system, and user auxiliary information output method that are capable of giving advice concerning proper diet in accordance with user conditions, or that make it easy for a user to know whether something is suitable to eat.

A user auxiliary information output device of a first aspect of the present invention comprises a user status information inputter for inputting status information (e.g., medical status information) of a user, and an advice outputter for outputting advice relating to user meals, using database information that is a combination of ingredients and cooking methods, obtained in accordance with the status information.

A user auxiliary information output method of a second aspect of the present invention comprises, inputting status information of the user, and outputting advice relating to meals to the user based on database information that is a combination of ingredients and cooking methods, obtained in accordance with the status information.

A non-transitory computer-readable medium of a third aspect of the present invention, storing a processor executable code, which when executed by at least one processor, performs a user auxiliary information output method, the user auxiliary information output method comprising: inputting diagnosis and examination information of a user, inputting status information of the user, and outputting advice relating to diet of the user based on the diagnosis and examination information and the status information.

### BRIEF DESCRIPTION OF THE DRAWINGS

FIG. 1A is a block diagram showing a user auxiliary information output system of one embodiment of the present invention, FIG. 1B is a block diagram showing internal structure of an information terminal of the user auxiliary information output system of one embodiment of the present invention, and FIG. 1C is a drawing showing one example of medical care food data in large hospitals and nursing facilities, for the user auxiliary information output system of one embodiment of the present invention.

FIG. 2A and FIG. 2B are flowcharts showing operation of a chatbot, in the user auxiliary information output system of one embodiment of the present invention.

FIG. 3 is a flowchart showing operation of information acquisition, in the user auxiliary information output system of one embodiment of the present invention.

FIG. 4 is a flowchart showing operation to determine advice content and reference period for advice completion, in the user auxiliary information output system of one embodiment of the present invention.

FIG. 5 is a flowchart showing operation for dietary advice, in the user auxiliary information output system of one embodiment of the present invention.

FIG. 6 is a flowchart showing another operation for dietary advice, in the user auxiliary information output system of one embodiment of the present invention.

FIG. 7A and FIG. 7B are flowcharts showing operation for display of providable information, in the user auxiliary information output system of one embodiment of the present invention.

FIG. 8 is a flowchart showing operation of a portable terminal, in the user auxiliary information output system of one embodiment of the present invention.

FIG. 9 is a drawing showing an example of data stored in a database (DB section), in the user auxiliary information output system of one embodiment of the present invention.

FIG. 10 is a flowchart showing operation of a CPU of a medical appliance, in the user auxiliary information output system of one embodiment of the present invention.

FIG. 11 is a flowchart showing operation of collecting data of training images, in the user auxiliary information output system of one embodiment of the present invention.

FIG. 12 is an illustration showing an example of displaying dietary advice on a portable terminal, in the user auxiliary information output system of one embodiment of the present invention.

FIG. 13 is a drawing for describing necessary conditions for dietary advice, in the user auxiliary information output system of one embodiment of the present invention.

### DETAILED DESCRIPTION OF THE PREFERRED EMBODIMENTS

An example where the present invention has been applied to a user auxiliary information output system will be described in the following as one embodiment of the present invention.

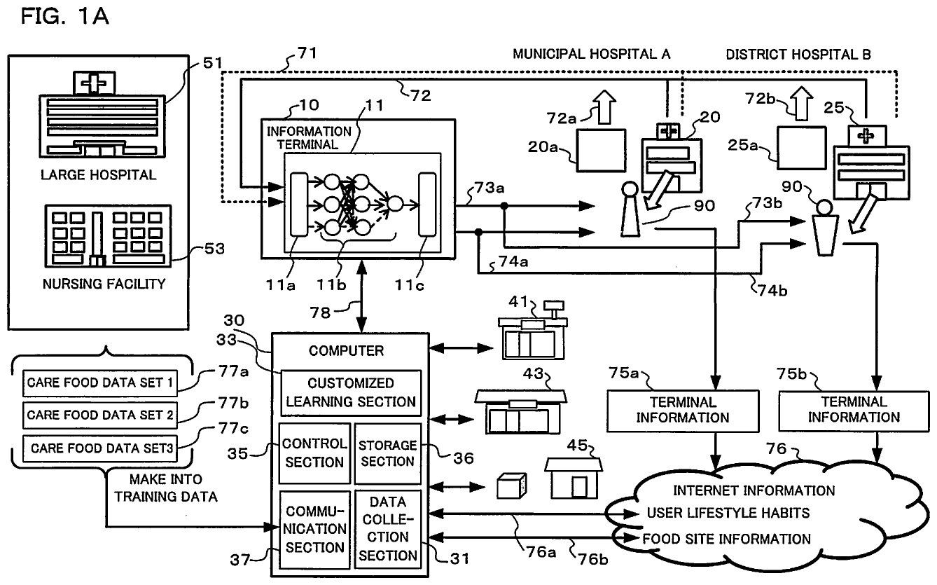

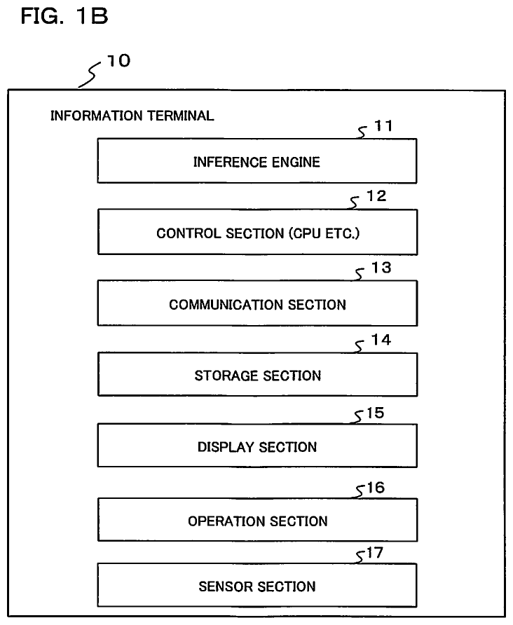

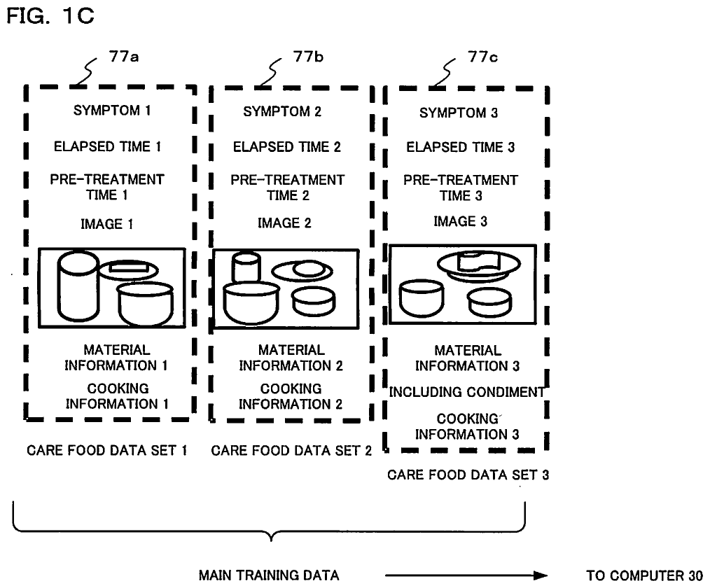

Referring to FIGS. 1A and 1B, an information terminal 10 is possessed by the user, and here is assumed to be a smartphone or a wearable terminal that is a peripheral device of the smartphone, or a portable terminal (which may also include a wearable terminal that can be used independently). In the case of a smartphone or the like, it is possible for the information terminal 10 to move together with the user, as the user is in possession of it. However, the information terminal 10 is not limited to a smartphone, and may be a stationary information terminal that does not necessarily move together with the user, such as a smart home appliance (including an AI speaker), digital home appliance, or personal computer, etc. The information terminal 10 may also have a function as a chatbot, and have an automated conversation program that uses artificial intelligence. Specifically, with a chatbot, a computer that has artificial intelligence functions can be communicated with (e.g., spoken to and heard from) instead of a human being.

User behavior can be known based on records of use and history, etc., of integrated circuit (IC) cards that can be used at shops and with means of transportation, and of installation locations of systems corresponding to these IC cards, and of user card usage, etc. It can therefore be said that corresponding devices at the time of IC card usage (system terminals) only function as a user's information terminal 10 at the time of IC card usage. Corresponding devices for those types of application also fall into the category of information terminals 10 of this embodiment. Besides this, the role of the information terminal 10 may be fulfilled by treating devices that constitute an electronic clearing system for other cards in the same way. In this way, if information terminals cooperate with system terminals, the system terminals can function as a part of the information terminals and function to collect rich information about users.

As shown in FIG. 1B, the information terminal 10 comprises a control section 12, communication section 13, storage section 14, display section 15, operation section 16, and sensor section 17, as well as the inference engine 11 shown in FIG. 1A. It should be noted that the inference engine 11 need not be provided within the information terminal 10, and may also be associated with a device (including a server, etc.) connected to a network, and at a location separated from the information terminal 10. There may also be cases where a terminal of an electronic fund transfer system is connected by a network to a control section away from the terminal, or cases where the terminal and control section are prepared in a dispersed fashion. These cases are also considered to have a control section. There may be cases where a portable terminal such as a smartphone only controls a user interface section, and other sections are associated with a control section on the cloud. With this application, this is represented as a control section 12 of a terminal, including associated units. Also, some functions of the control section 12 and functions of the storage section 14, etc., may also be dispersed among devices that are capable of communicating by means of a communication section such as the cloud or an external computer.

Referring to FIG. 1A, the inference engine 11 comprises an input layer 11a, intermediate layers (neural network) 11b, and an output layer 11c. The inference engine 11 performs inference using an inference model that has been generated by a computer 30. This inference engine 11 is input with user status information 71 indicating state of the user 90 of a municipal hospital A 20 or district hospital B 25, and diagnosis and examination information 72 of a medical facility or examination agency, etc., and infers advice relating to the users diet, to the user, such as information that is a combination of diet and cooking methods, etc. This advice is displayed to the user 90 as simple guides 73a and 73b, and special guides 74a and 74b. Also, advice using an image of a meal is possible. The inference engine 11 may infer for images of meals retrieved by internet.

It should be noted that the inference engine 11 constitutes a neural network that also has memory, in a circuit block provided on an AI (Artificial Intelligence) chip such as a CPU, GPU, DSP, etc. This inference engine 11 is connected to a network or the like associated with a medical facility, and/or examination agency, etc., and in this embodiment it is assumed that there will be cases where the control section 12 can be used in cooperation with these institutions.

Referring again to FIG. 1B, the communication section 13 has a communication circuit, and can perform communication with the computer 30, information processing devices such as computers in medical facilities and examination agencies, etc., such as the municipal hospital A20, and the district hospital B25, a computer and a server for an in-hospital system. Specifically, the communication section 13 can receive an inference model 78 from the computer 30. Also, the communication section 13 can receive user status information 71 and diagnosis and examination information 72 from the medical facilities such as the municipal hospital A 20 and district hospital B 25, and from inspection agencies, etc. The diagnosis and examination information 72 includes medical appliance information 72a and 72b for the medical facilities and examination agencies, etc. These items of information are used when obtaining advice for the user relating to diet by means of inference. Also, this type of function may also be possessed by a cloud-based computer 30, enabling cooperation between each device, each terminal, and each system.

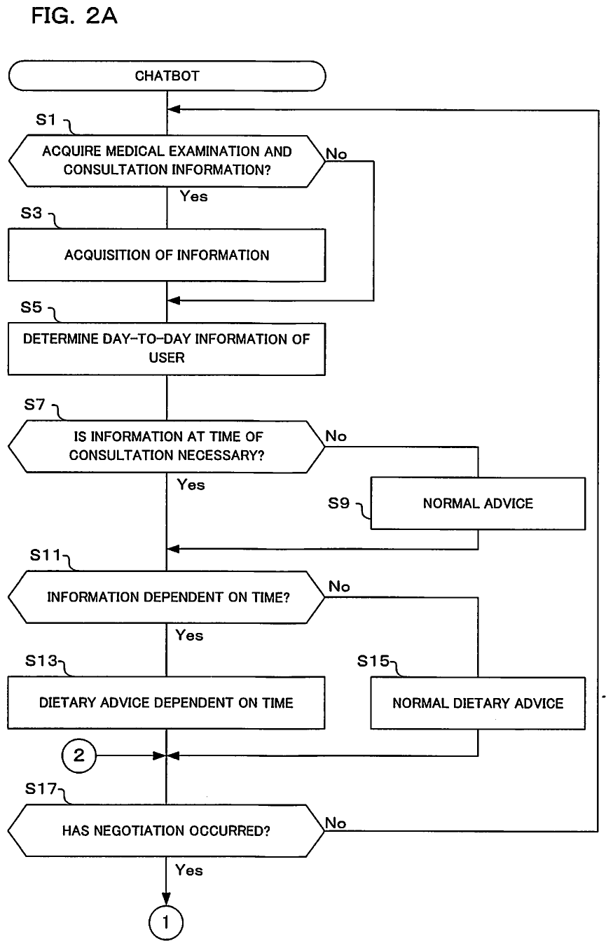

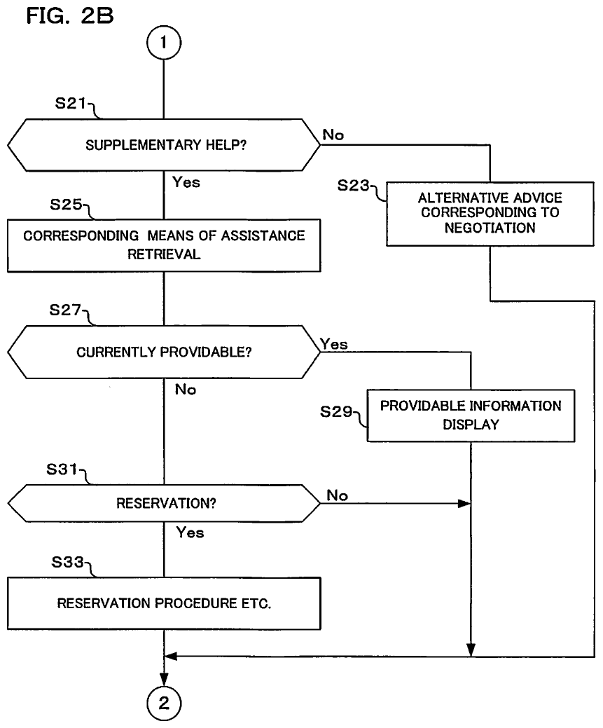

The communication section 13 may also function as a user status information input section (user information inputter) for inputting status information of the user (refer, for example, to S5, etc., in FIG. 2A). Status information of the user that is input by the user status information input section (user information inputter) is at least one of, for example, location of the user, current image of the user themselves, time since the user received a diagnosis, current time, user profile, user lifestyle habits, user medical history, user's food and/or beverage ingestion history, etc.

The communication section 13 may also function as a diagnosis information input section (diagnosis information inputter) for inputting information that was obtained at the time of user diagnosis (refer, for example, to S3 in FIG. 2A etc.). The communication section 13 functions as a diagnosis and examination information input section (diagnosis and examination information inputter) for inputting diagnosis and examination information of the user (refer, for example, to S3 in FIG. 2A, etc.) Diagnosis information relating to diagnosis, and diagnosis and examination information, is not limited to information that was acquired when a doctor, etc., consulted with the patient, and is used with a wide meaning, such as information that was acquired at the time of an examination that was performed for the purpose of diagnosis, and information at the time a booking was made at a medical facility or examination agency for the purpose of receiving a consultation or examination. The diagnosis information input section (diagnosis information inputter) is input with results of user diagnosis and images that have been obtained (refer, for example, to S3 in FIG. 2A). Diagnosis results and images that have been obtained may include, for example, endoscope images, chest x-ray photographs, MRI (Magnetic Resonance Imaging) images, CT (Computed Tomography) images, etc. These images constitute suitable information at the time of identifying symptoms and determining results of treatment.

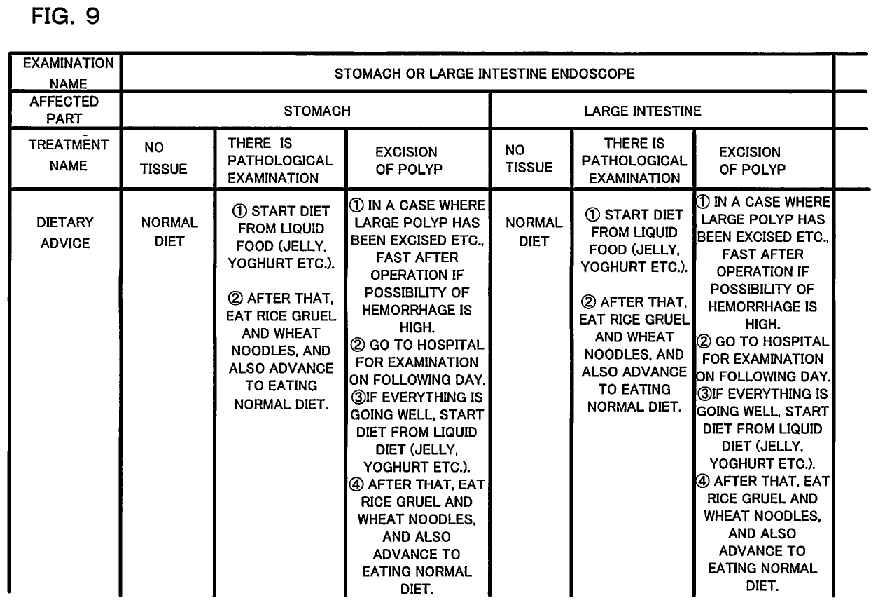

The storage section 14 is an electrically rewritable non-volatile memory, and stores various data. Programs that are used by a CPU within the control section 12 may also be stored. The storage section 14 also stores information such as the diagnosis and examination information 72 and user status information 71 that has been received using the communication section 13. The storage section 14 may also have a database in which dietary advice corresponding to diagnosis and examination results, etc. is stored (refer to FIG. 9). It should be noted that similar functions may also be fulfilled by the storage section 36 of the computer 30, and the technology of this embodiment may also be realized as a cloud service.

The storage section 14 functions as a storage section that stores user diagnosis results as information (refer, for example, to S3 in FIG. 2A). The storage section may also store images that were acquired at the time of diagnosis (for example, endoscope images, chest x-ray images, MRI images, and CT images), as diagnosis results for the user (refer, for example, to S3 in FIG. 2A, and to FIG. 9). This function may be acquired by communication with a computer for an in-hospital system (not illustrated, for simplicity) for managing medical appliances 20a, 25a, which will be described later, or alternatively, may be realized by referencing this computer.

The display section 15 has a display, and can display advice relating to diet that has been inferred by the inference engine 11. As well as being inferred, this advice relating to diet may also be displayed based on search results from a database of advice relating to diet in the storage section 14. Also, the display section 15 can display various information such as various modes of the information terminal 10 and icons for input. It should be noted that in a case where the information terminal 10 functions as a chatbot and speech is used, a microphone, speaker, and voice recognition circuit are provided. These components are not necessary if the chatbot is text-based.

The operation section 16 may include operation members such as a touch sensor that is directed towards the display, and various buttons. Using the operation section 16, it is possible for the user to perform instruction operations in order to issue various instructions to the information terminal 10.

The sensor section 17 has various sensors, and processing circuits, etc., for processing outputs of the various sensors. As the various sensors there are, for example, a GPS (Global Positioning System) for detecting position of the information terminal 10, and an imaging section for acquiring images. Since a user moves while possessing the information terminal, it is possible to determine a position of the user utilizing the fact that the GPS is provided. Also, it is possible to acquire images of the user themselves using the imaging section, and it is possible to determine health status of the user by analyzing these images. It is also possible to obtain information relating to diet if the user acquires images of meals they have eaten, using the imaging section.

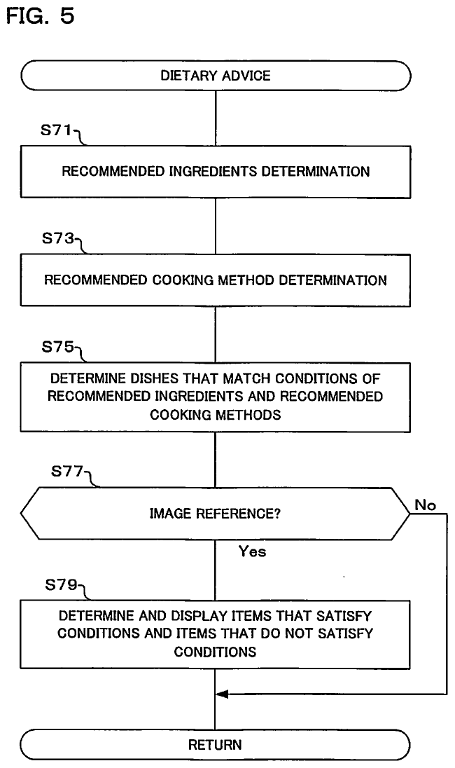

The imaging section (image sensor) within the sensor section 17 functions as an imaging section (image sensor) for inputting ingredients or food that has been cooked as images (refer, for example, to S77 and S79 in FIG. 5). It should be noted that in a case where the information terminal 10 does not have an imaging section, image data may be input by means of the communication section 13 etc. In this case, the communication section 13, etc., may function as an image input section (image input circuit), for inputting images of ingredients or food that has been cooked.

Also, if the information terminal 10 is configured so as to be associated with a smartwatch, it is possible to acquire vital information such as number of steps, pulse rate, and blood pressure, etc., of the user. The information terminal 10 may also have a sensor for measuring these items of information.

The control section 12 is an IT unit or processor and may include a CPU (Central Processing Unit), memory, hard disk drive (HDD), CPU peripheral circuitry, etc. There may be one such processor, or there may be a configuration comprising a plurality of chips. The CPU implements the overall control of the information terminal 10 by controlling each of the sections within the information terminal 10 in accordance with programs stored in memory. Each section within the information terminal 10 may be realized by software control using a CPU. The previously described communication section 13, etc., may also be arranged within the processor constituting the control section 12.

Also, the control section 12 determines status of the user based on user status information 71 of the user 90 that has been received by the communication section 13. The control section 12 functions as a user status determination section for determining status of the user (refer, for example, to S5 in FIG. 2A). The user status determination section determines status of the user based on at least one among location of the user, current image of the user themselves, elapsed time since the user received a diagnosis, and current time (refer to S5 in FIG. 2A).

The control section 12 also functions as an advice output section for outputting advice relating to diet of the user, obtained in accordance with information relating to diagnosis and status information of the user (refer, for example, to S9, S13, and S15 in FIG. 2A, and to S29, etc., in FIG. 2B). The control section 12 also functions as an advice output section (advice outputter) for outputting advice relating to diet of the user based on diagnosis and examination information and status information (refer, for example, to S9, S13, and S15 in FIG. 2A, and to S29, etc., in FIG. 2B). Advice relating to diet may also be created based on information that is a combination of ingredients and cooking methods. It should be noted that diet refers to things that are prepared (e.g., cooked, mixed, tossed, combined) using ingredients (assumed to be ingredients that are hunted, gathered, or cultivated, such as meat, fish, vegetables, grain, etc.) and cooking methods, or other methods of preparation.

The control section 12 functions as an advice output section (advice outputter) that outputs advice relating to shops or services that are capable of providing food that is suitable for the user in accordance with status information, in association with information possessed by point of sale (POS) systems of shops or services (refer, for example, to S9, S13, and S15 in FIG. 2A, and S29 in FIG. 2B). Specifically, the advice output section (advice outputter) outputs advice relating to diet of the user by causing coordination of database information and information available at a POS system. In this case, part of a database in which ingredients and food preparation (e.g., cooking) methods have been combined may be stored in a POS system, and part of that database may be stored within an application. Also, the control section 12 functions as an advice output section (advice outputter) that outputs advice relating to diet of the user, using data information that is a combination of ingredients and cooking methods, obtained in accordance with status information (refer, to S9, S13, and S15 in FIG. 2A, and to S29 in FIG. 2B, etc.). The information by POS system includes image information of product (ingredient, food and drink, etc.) according to trade name.

In order for the advice output section to output advice relating to diet, a database, etc., is prepared in which, for every item of food and drink, their ingredients (including, for example, starch, protein, grains, fruits, vegetables, spices and aromatics, etc., flavor enhancers, oil that is used, presence or absence of heating, whether it is fresh, whether it is fermented, whether it is brined or salted, etc.) and food preparation (e.g., cooking) methods, or amounts for calories, salt content, or sugar, etc. are specially managed, and determination of advice is possible using the database, by dividing into ingredients, food preparation method, and seasoning (for example, salt content, sugar, calories, condiments, toppings, etc.). If a result of this determination is that the user is permitted to eat respective ingredients, then by similarly considering that the user is also permitted to eat items that are cooked using these ingredients, it is possible to select safe meals and drinks for each case. Also, this database may store representative image information and recipes, etc., in association with each other, and it is possible to search using images, and it is possible to search ingredient information and cooking information using recipes. If there is a recipe, then a food preparation (e.g., cooking) method is known, and it is possible to determine material change, etc., between a case where there is heating, cooking via acids such as ceviche’, fermentation, curing, etc., and a case where there is no heating, acid cooking, fermentation, curing, etc.

Also, this database may also classify, for every completed meal or drink, whether it is healthy, or whether it is not healthy in general, or for a user having particular medical conditions. However, with this embodiment, since detailed information is desired for every case and body composition, classifications such as ◯◯ case (that is, OK) and ΔΔ case (that is, NG or not good) will be provided for finished products of meals and beverages. However, with this method, management becomes difficult every time a new commodity is added because the amount of information might increase too much. Therefore, making classifications such as ◯ ◯ case OK, ΔΔ case NG for every finished product of meal and beverage may be advantageous in that such classifications help the system in simply determining many finished meal and beverage products as it can be applied to a wider range by examining their respective ingredients.

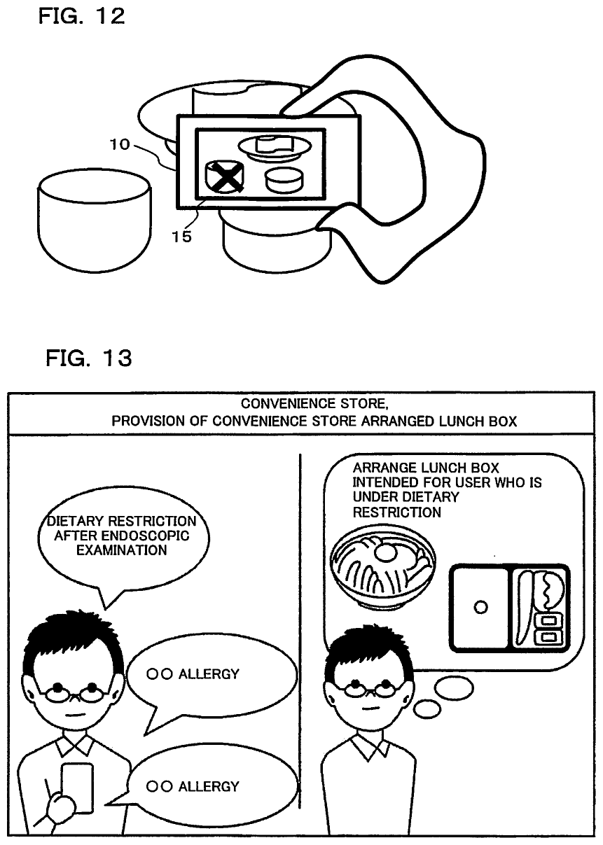

Also, the control section 12 functions as an advice output section (advice outputter) that outputs advice for displaying suitability of the user eating something, in accordance with position of a food item within an image, for images that have been input using the image input section (refer, for example, to S77 and S79 in FIG. 5, and to FIG. 12). Specifically, the advice output section (advice outputter) outputs advice relating to diet of the user for every position of food and ingredients within an image that has been taken of dietary items that should be ingested by the user, so as to be able to display suitability for the user to eat. Images will generally be arranged on a two dimensional space, and there will be convergence of visible information for each position in that image, and because of this, it is inherently easy for the user to understand, whereby the user can make an informed determination of whether or not to eat a given item of food at a glance. Utilizing this characteristic, it is also easy to perform annotation and determination such as what is at which position within a screen at the time of image display, and there is a state where it is easy to use a lot of images as training data for artificial intelligence (AI), or a state where it is easy to use in input for image determination AI. Alternatively, or in addition, a machine may be trained using sets of training data including food ingredients, food preparation methods, user information, and/or user diagnosis or examination information as inputs, and including acceptable (O) or unacceptable (X) as outputs.

With this embodiment, utilizing this characteristic that images have, it becomes possible to determine whether something is good or bad for the diet of the user based on what type of food is at different positions within an image displayed on a screen. For example, the control section 12 can check what type of food or drink is where within a screen, or where container is, or what is contained in that container using images in a database that has been created from similar image retrieval and those images that have been retrieved, and ingredients and cooking methods at the time similar meals are created. Recipes may also be respectively determined. It is then determined whether each of different food and/or beverage items in different sections within a screen is suitable, or not suitable, to be ingested by the particular user. These determinations mean that meals can be easily inferred using AI. That is, if many images including meals are collected, and teaching data is made from this image data by annotation (for example, “suitable” or “unsuitable”) for the meals, an inference model (inferring whether or not a meal is suitable) can be made using this teaching data. This point will be described again using FIG. 12, etc.

Also, the control section 12 functions as an advice output section (advice outputter) that outputs advice in accordance with information that has been obtained, at the time of user diagnosis (refer, for example, to S9, S13 and S15 in FIG. 2A). This advice output section may also output advice using user status information in addition to the diagnosis information that was obtained at the time of diagnosis.

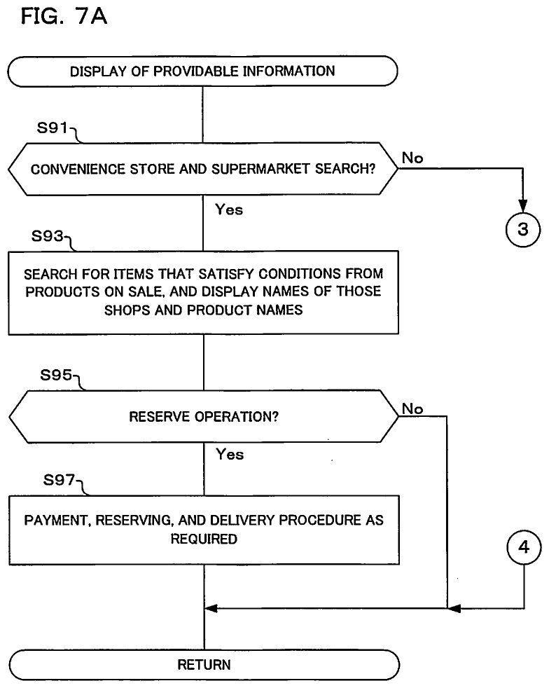

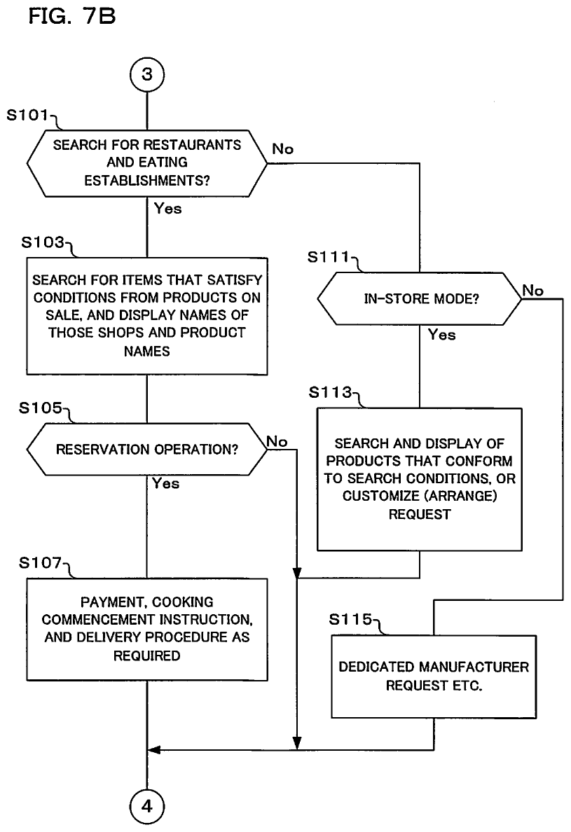

Also, advice relating to diet that is output by the advice output section (advice outputter) (control section 12) includes food shop information or service information for food shops or services that are capable of supplying ingredients and/or dishes, in accordance with ingredients and cooking methods (refer, for example, to S29 in FIG. 2B, and S93 in FIG. 7A). Also, advice relating to diet that is output by the advice output section (advice outputter) (control section 12) may include food shop information or service information for shops or services that are capable of supplying meals (meals including desserts, and drinks) in accordance with ingredients and cooking methods (refer, for example, to S29 in FIG. 2B, and S193 in FIG. 7B). Here, shop information and service information may include name and contact details of a restaurant, and as service information there are name and contact details of a food delivery service, etc. Also, advice that has been output by the advice output section (advice outputter) may be cleared with doctors, nutritionists, and health care professionals before meals are taken (for example, a step of confirming with a doctor or the like may be provided at a point in the process before or after S13 and S15 in FIG. 2A).

The control section 12 may also determine whether or not there is a schedule for a user to receive an examination, based on status information that has been input by the user status information input section (refer, for example, to S3 in FIG. 2A). The control section 12 may also function as an advice output section (advice outputter) that provides advice relating to meals before the day an examination is scheduled (refer, for example, to FIG. 2A). For example, a user may be instructed to fast for 12 hours before a blood test.

The advice output section (advice outputter) outputs advice based on characteristics of results of user diagnosis, and examination results within images that have been acquired (refer, for example, to S13 and S15 in FIG. 2A). For example, in a case where endoscope images have been obtained as images, advice is output as a function of the size of polyps within those images. The advice output section (advice outputter) may also change content of advice relating to meals, and/or a period in which advice relating to meals is output, in accordance with information relating to user diagnosis (refer, for example, to S13 and S15 in FIG. 2A). For example, a period in which advice is output may be changed as a function of the size of polyps obtained as a result of diagnosis.

It should be noted that here procedures for an endoscope have been described, but in a case where other examinations and procedures have been performed then advice that is output may also be changed in accordance with results of those examinations and procedures. For example, with a dental region, there are cases where it temporarily becomes impossible to use the mouth or teeth, depending on conditions at the time of treatment, and also with oral surgery and dermatology there is similarly change in recommended items for each of symptoms and treatment. There are also cases such as where various examinations, for example, collection of blood, do not go well, and cases where examinations are tried again after waiting for a while. At a time when symptoms are acute, there will be cases where measures are taken after the patient has stabilized, and in this case also, it is better to change the way in which advice is output after diagnosis and examination.

With a time at which the user received a diagnosis as a reference, the advice output section (advice outputter) may change advice relating to meals of the user in accordance with period from that time. (for example, FIG. 2A). A time at which diagnosis was received is not limited to the time when a doctor, etc., consulted with the patient, and may also be a time at which a medical examination or medical procedure was performed, for example. The period is not limited to being after a reference day, and with an examination day, medical procedure day, or consultation day as a reference may be before that reference day. Also, in a case where the information terminal 10 has an image input section (or imaging section) for inputting ingredients, or items having food, recipes and proportions arranged, as images, the advice output section (advice outputter) may display how suitable it is for the user to eat the items in the images that have been input by the image input section (imaging section) (refer, for example, to S77 and S79 in FIG. 5, and to FIG. 12).

The advice output section (advice outputter) may also provide a meal search service, a meal ordering service, and/or a restaurant reservation service to shops and service providers that provide ingredients and meals and drinks that conform to advice relating to diet, in accordance with advice (refer, for example, to S31 and S33 in FIG. 2B). The information terminal 10 performs searching, ordering, and/or reservation relating to user meals, using information that is a combination of ingredients and recipes, obtained in accordance with status information (refer, for example, to S31 and S33 in FIG. 2B). It should be noted that this ordering and reservation may also be performed by computer 30 or another computer or server, in place of the information terminal 10. The advice output section (advice outputter) may prepare a plurality of items of advice relating to user meals obtained in accordance with diagnosis results and/or determination results for user status, and may sequentially display these items of advice in accordance with a priority order (refer, for example, to S23 in FIG. 2B).

It should be noted that the advice mentioned above is not only for the person in question, and it may also be possible to display etc., and output, to information terminals of homes, partners, and auxiliary personnel who prepare meals for that person. In this case, the same information may also be transmitted to information terminals of other people that the user has set, and it should be possible to access information from information terminals those of the people.

The municipal hospital A 20 and district hospital B 25 are medical and examination agencies where users 90 undergo medical consultations, examinations, and/or procedures. When a user consults a medical or examination agency, and in the case of a user registering, it becomes possible for the information terminal 10 to acquire various information from the medical and examination agencies, etc., by inputting an application program for the user to the information terminal 10. If the information terminal 10 of the user becomes capable of acquiring information from the medical and examination agencies, etc., it will be possible for the information terminal 10 to acquire diagnosis and examination information 72 of the user 90. In the medical and examination agencies etc., it may be possible for the information terminal 10 to acquire diagnosis and examination information 72 such as diagnosis results, by making appointments to receive services such as diagnosis and examination results etc.

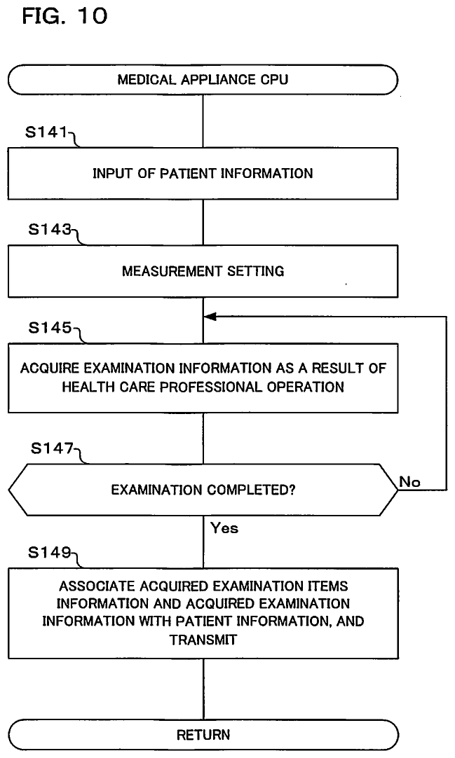

Also, medical appliances 20a are arranged in the municipal hospital A 20, and medical appliances 25a are arranged in the district hospital B 25. The medical appliance information 72a and 72b of the medical appliances 20a and 25a may also be transmitted to the information terminal 10 together with diagnosis and examination information 72. Also, the medical appliances 20a, 25a need not be standalone devices, but may be connected to a medical server within medical and examination agencies, and this server may transmit diagnosis and examination information 72 to the information terminal 10. Information relating to medical treatment and health, such as medical appliance information, and diagnosis and examination information, is also collectively called “medical information.” Detailed operation of medical appliances 20a and 25a, etc., will be described later using FIG. 10.

The medical diagnosis, examination, and/or procedure information 72 may also include time and date information for an examination and/or treatment time for medical and examination agencies such as the municipal hospital A 20 and district hospital B 25, as one example of information on initial information output. From this time point, it is assumed that change in the user's condition occurs with time, such as natural healing or deterioration depending on conditions. Also, status information 71 of the user may also include current time, in order to obtain advice that is appropriate at that point in time. As a result, it is possible to produce appropriate advice in consideration of change in condition that is expected to occur with time, at a point in time after the above-described initial information output.

Also, the diagnosis and examination information 72 may also be results of diagnosis and/or treatment received by the user at a medical facilities. The chronological change in user condition may vary depending on those results. There may be cases where progress transitions to a favorable situation as a result of respective diagnosis and treatment, cases where this is not the case and no change occurs, or unfavorable change occurs, and cases where those time changes are included, but simple determination of the user's condition at a given point in time is not possible, etc. Since there are such differences, it is better if there is information for determining such differences, and examination data of bio-information of medical facilities and/or examination agencies, and/or data on specimen test results may also be included. Results of diagnosis and/or treatment may be items resulting from having answered one or more inquiries, symptoms, possible disease names, disease names that have been defined, and documents of types, content, and effects of treatments, into data, and may also be in the form of putting examination results into writing. Also, results of treatment may also include information on condition of parts of the body that were treated (names of the body parts, degree of invasion, size, position, condition, etc.).

The previously described inference engine 11 may be used to perform inference using an inference model in order to output simple guidance if user status information 71 and diagnosis and examination information 72 are input. When the user 90 has undergone a medical examination, a medical treatment, etc., at a medical facility or examination agency such as the municipal hospital A 20, this simple guidance is supplementary advice, based on medical information at that time, for when the user 90 performs activities outside a hospital. For example, after having received a vaccination shot, etc., there will be cases where, depending on the vaccine that has been administered, it is advisable to go to sleep early, and in this case dietary advice is given so as to discourage the intake of caffeine. Also, in a case where polyps have been found when undergoing an endoscopic examination of the colon, advice such as encouraging the eating of something that is easily digestible is given, and advice is given to avoid eating things like seaweed, sesame, onions, etc., that may irritate the user's colon.

Also, in addition to medical information, the inference engine 11 outputs special guidance 74a and 74b based on information such as lifestyle habits of the user, and provides the special guidance to the user 90. The previously described simple guidance 73a and 73b is advice that is given in association with a medical diagnosis, examination, and/or procedure for only a specified duration from a specified time, with a specified time when the consultation/diagnosis, examination, and/or procedure was undertaken as a reference, in a municipal hospital A 20, etc. By contrast, the special guidance 74a and 74b assumes that advice is given in a case where the user 90 wants to monitor their life (health), either before or after medical consultations, examinations, and/or procedures such as endoscopic examination, etc. For example, for people that have a lot of neutral fat, advice is given so as to avoid foods with a lot of fat, such as butter, cream, beef, and pork, etc., foods having a lot of carbohydrate, such as fruit, honey, confectionary, and juices, and alcoholic drinks such as sake. An inference model that analyzes user behavior in this way, and makes this analysis a trigger to determine what types of effects are brought about, may be called Mokkat (Multiple organized key knowledge active trigger).

In the case of giving the above described special advice 74a and 74b, the information terminal 10 acquires information relating to condition of the user 90 (terminal information, etc., 75a and 75b), and information is transmitted to the computer 30 as lifestyle habit information of the user. The terminal information, etc., 75a and 75b may also include information such as where the user 90 is, what they have purchased and where, and what they have eaten, etc. The terminal information, etc., 75a and 75b may also store main points of vital information and health diagnosis results. Information on refrigerators and air conditioners, heaters, ventilation, local temperature, local humidity, local air quality, etc., of the user may also be referred to. In order to acquire these items of information, the information terminal 10 may be provided with GPS, and acquire position information. The information terminal 10 may also be provided with an imaging section and an image analysis section, and acquire information by analyzing items the user 90 has purchased and their behavior, etc. Further, information relating to purchased items may also be acquired from payment information for IT use, such as credit card payments, purchase made via online applications or apps, etc.

The computer 30 operating in cooperation with the information terminal 10 acquires data that cannot be found in these types of hospitals and nursing facilities, and generates training data based on this data. Living or lifestyle information of users, such as what effects these type of eating habits will have, is collected in the computer 30. Training data in which data that relates to lifestyle habits of this user has been acquired constitutes an upgraded version of training data.

Also, a large hospital 51 and nursing facility 53 provide care food to patients and residents every day. As shown in FIG. 1C, care food data sets 77a to 77c of the care food includes symptoms of the patients and residents (symptoms 1 to 3 in FIG. 1C), elapsed time (elapsed times 1 to 3 in FIG. 1C), pretreatment time (pretreatment times 1 to 3 in FIG. 1C), image (images 1 to 3 in FIG. 1C), material information (material information 1 to 3 in FIG. 1C), and cooking information (cooking information 1 to 3 in FIG. 1C). If there is a large hospital (including a place of treatment, or long term hospital where the user lives) 51 or nursing facility 53, there is information on what type of symptoms there are, and what type of food menus are effective, for every age and gender that have had given medical diagnosis, examinations, treatments and procedures, at given times in the past. Then, since it is possible to make a care food data set if these items of information are arranged, this data set is collected, and ingredients and cooking methods that are included in a set in this data are made into main training data. Training data should have information such as image data, and protein, carbohydrate, fat, calories, salt content, fiber content, etc.

This training data can also be used as a cooking database. FIG. 1C includes image examples of complete sets of dishes corresponding to a single meal (images 1 to 3). However, since it is more difficult for the user themselves to cook and prepare food than it would be if prepared at a specialist facility, training data may be respectively created by being divided into content for each vessel, such as individual dishes, etc. That is, the examples shown in FIG. 1C include conceptual sections, each with a portion of a certain type of food, and in actual fact it becomes more practical to arrange for every image of every dish, bowl, and vessel. However, with single items there may be cases where there is a possibility that necessary nutrients and calories or some other relevant nutritional information will be missing, and it is also possible to use images of a complete set of meals that make up for these missing nutrients or calories or some other relevant nutritional information.

In this case, since dietary intake with the same meals also constitutes important health management indices, it may be made possible to estimate amounts of food (weight, etc.) for every size of container, tableware, or tray, etc., and these items of information may also be separately stored in a database. For example, when determining whether or not specific nutrients and calories are sufficient, it may be made possible, for training data for a single set of meals, to use AI to determine insufficient side dishes (for example, nutrients and calories of that side dish), etc., and AI to determine excessive calorie intake due to overeating, etc.

By devising this type of configuration, it is possible to perform determination for what type of meals are appropriate for what type of case, and what stage of healing, and if stored in association with ingredients and cooking methods for those meals (in a case of making into training data, association of data in a format that can be subjected to annotation), it becomes possible to determine suitability for every meal served in respective vessels. There are also cases where a plurality of meals (having different ingredients, cooking methods and recipes) are served on the same plate. In this case, determination as to what materials and main meals are arranged at what position within a screen is performed based on color, texture, and borderlines of food within a vessel, etc., and it is possible to make databases and to create training data by respectively dividing information.

It should be noted that this training data and database may also be stored in association with representative image information and recipes, etc., so as to enable search based on images, and so as to enable search for ingredient information and cooking method information based on recipes. If there is a recipe, then a cooking method is also known, and it is also possible to determine material change etc., between a case where there is heating and a case where there is no heating.

Here there may be cases where training data is used in deep learning when creating an inference model to extract specified information B from specified information A (for example, image data). Training data is also written to information A in a case where learning is performed by annotating information B. However, if information B is associated with information A and stored in an arrangement such that mutual relationships between these items of information are understood and it is possible to retrieve information relating to information B from information A, or retrieve information relating to information A from information B (or C and D that are related), then since this can be represented as a database, ultimately, information constituting training data and information constituting a database can be considered substantially the same. Also, annotation information may be optionally selected.

Individual position information for ingredients and meals that are within an image displayed on a screen may also be stored together, and there may be a configuration such that it is possible to perform annotation for each of those positions within the displayed image. Temperature information and other data that cannot be known directly from an image may also be arranged at the same time. Alternatively, the terminal 10 may be provided with a sensor to sense temperature of the food. Also, relationships between affected parts and their symptoms, and each meal that has been described here, or ingredients and cooking methods and recipes for those meals, may also be made into a database, and this will be described later using FIG. 9. Also, since there is a possibility that various meals that can be completed by combining various ingredients and cooking methods will become too diverse due to a large number of permutations, a database breaking these meals down further into materials may also be prepared. A database broken down into each cooking method that includes seasoning, oil used, etc., may also be prepared. For example, in the case of a green salad, in addition to what type of cases lettuce will pose a problem for, taking dressing as an example of cooking method, it is possible to reflect differences such as that soy sauce is good as a dressing, and sesame oil is bad as a dressing. Naturally, it will also be possible to perform determinations such as that salad itself is bad, depending on body composition. Adopting the way a side dish was considered previously, it is possible to create AI to perform determinations such as something would be good with warm items. In this case, besides information as to whether something is good or bad for the body, what would be lacking if only that item was eaten may be stored, together with information on recommended side dishes, in a database.

The computer 30, that has been assumed to be a server, etc., is configured by a general purpose computer comprising a control section 35 for performing overall system control as required, or performing control by means of cooperation between other computers, servers and portable terminals etc. The control section 35 may be an information processing section such as a CPU, memory that stores programs, etc., and peripheral circuitry such as a communication circuit, etc. The computer 30 also has a data collection section 31 and a customized learning section 33. The data collection section 31 collects care food data sets 77a to 77c from the large hospital 51 and nursing facility 53 that were described previously, and at this time also collects information for protein, carbohydrate, fat, calories, salt content, etc., and creates main training data.

Also, the computer 30 has a communication section 37. Data collection and data output are communicated to each terminal, server, and computer, etc., via the communication section 37 by means of the Internet or a network, and it is possible to perform communication between the information terminal 10, and information processing devices such as computers in medical facilities and examination agencies such as the municipal hospital A 20 and the district hospital B 25, and computers and servers for in-hospital systems. It is possible to receive user status information 71 and diagnosis and examination information 72 from the medical facilities and examination agencies such as the municipal hospital A 20 and district hospital B 25, etc. Medical appliance information 72a and 72b for the medical facilities and examination agencies, etc., is included in the diagnosis and examination information 72.

It may also be assumed that the computer 30 may be implemented as a server, etc., that administers cloud service general control. It is not necessary for this type of server to be a single unit, and as well as fulfilling a function of assuming cooperation with a server having various roles, some functions of the user information terminal 10 may be assumed, or information from those functions may be obtained, and a specified service may also be made possible by further adding other information to this information. For example, the inference engine 11 that was described as possibly being built into the inference engine 11 may also, or alternatively, be provided within the computer 30, or alternatively may be provided within a computer (server) that cooperates with the computer 30, and it is also possible for the information terminal 10, as it were, to operate so as to have that function, as a result of cooperation between the information terminal 10 and the computer 30. That is, the various information that has been described above, used when obtaining advice relating to user diet by inference, can be collected in a cloud database, and it is also possible to have a cloud service that uses these items of information. By developing assumed services, it is also possible to change sharing of roles and cooperation between each device.

It may also be possible for the computer 30 to cooperate with other businesses, such as performing control in cooperation with commodity ordering and delivery systems of, for example, the home delivery industry, food service industry, retail businesses, restaurants, delivery industry, etc. By cooperating with these systems, it becomes possible for a control section of the information terminal 10 or the computer 30 to also realize services that satisfy various meal needs by accepting user orders. Also, even if the user themselves does not perform ordering, in a case where there is consent with the user by virtue of a contract, user information such as has been described above is input to control sections of shop and service systems, and schemes to perform services such as pre-preparing meals, food, desserts, and drinks suitable for a user, and ingredients and cooking methods for these meals, etc., are also possible. It is possible to configure a system whereby user status is determined, ingredients, those ingredients and cooking, and principal meal content such as arrangements of dishes, desserts, and drinks, that are suitable for that status, are inferred, and this information is provided to the user.

Accordingly, as a user auxiliary information output device of this embodiment, this computer 30 may be assumed, and there may be cooperation with an information terminal (portable terminal) that is actually operated personally by the user. At this time, there are a user status information input section for inputting status information of the user (here, the communication section 37, for example), and an advice output section for outputting advice relating to shops or services capable of supplying meals that are suitable for the user, in accordance with the status information, by means of coordination of information possessed by POS systems of the shops or services (here, the control section 35, for example).

Also, by means of the above described cooperation, control sections that control associated industry and corporate systems output information to procure corresponding ingredients, and output information on ingredients and cooking methods to associated factories or commercial kitchens, and products conforming to those specifications may be delivered by a specified time. This specified time must be before a time when the user will want to purchase ingredients and products, etc. A time when the user will want to purchase things may be a time until passing or arriving at that shop, or may be at the time of breakfast, lunch, or evening meal, or correspond to another time. Being able to offer these types of goods is preferably achieved by enabling a control section to provide information to a user by means of information cooperation and service cooperation.

Also, the data collection section 31 may be used to generate an upgraded version of training data based on information from Internet information 76. The Internet information 76 has, for example, lifestyle habit information 76a and cooking site information 76b of the user. The lifestyle habit information 76a is collected from terminal information, etc., 75a and 75b. Also, cooking site information is, for example, photographs of cooking that have been published on the Internet (for example, Cookpad (registered trademark)). Also, since cooking size information also includes ingredients and cooking methods, it is important to perform learning using these items of information. Training data is generated by attaching annotation to images, and this attachment of annotation may be performed by a person, and may be attached automatically by a computer.

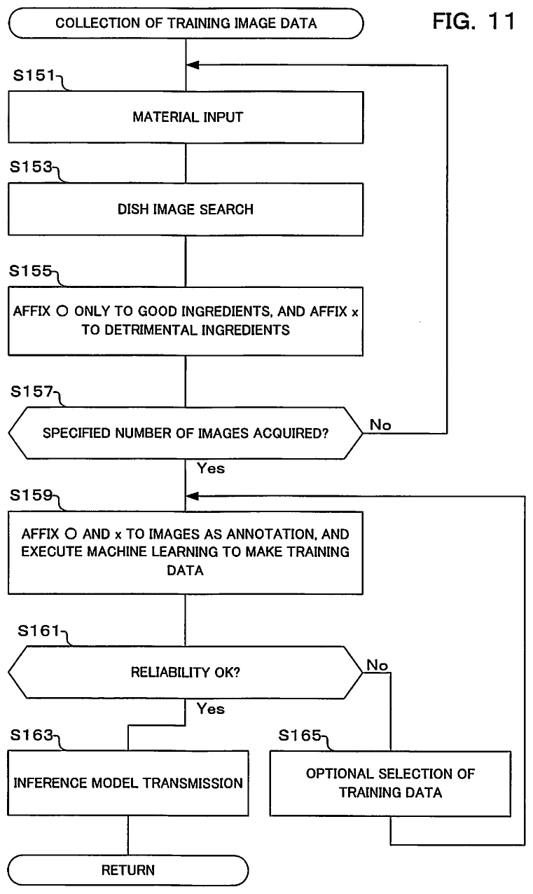

The customized learning section 33, similarly to the inference engine 11, comprises an input layer, intermediate layers (neural network), and an output layer. By performing machine learning using training data that has been collected by the data collection section 31 parameters for strength of interconnections of each layer of the intermediate layer (neural network) are obtained, and an inference model for performing simple guidance and special guidance is generated. The inference model for simple guidance and special guidance that has been generated here is transmitted to the information terminal 10 via the communication section 37 within the computer 30, and stored in the inference engine 11. Details of operation for data collection and learning for inference model creation will be described later using FIG. 11.

Here, deep learning will be described. “Deep Learning” involves making processes of “machine learning” using a neural network into a multilayer structure. This can be exemplified by a “feed-forward neural network” that performs determination by feeding information forward. The simplest example of a feed-forward neural network should have three layers, namely an input layer constituted by neurons numbering N1, an intermediate later constituted by neurons numbering N2 provided as a parameter, and an output later constituted by neurons numbering N3 corresponding to a number of classes to be determined. Each of the neurons of the input layer and intermediate layer, and of the intermediate layer and the output layer, are respectively connected with a connection weight, and the intermediate layer and the output layer can easily form a logic gate by having a bias value added. These connection weights are adjusted and/or determined while the neural network is trained with a set of training data.

While a neural network may have three layers if simple determination is performed, by increasing the number of intermediate layers it becomes possible to also learn ways of combining a plurality of feature weights in processes of machine learning. In recent years, neural networks of from 9 layers to 15 layers have become practical from the perspective of time taken for learning, determination accuracy, and energy consumption. Also, processing called “convolution” is performed to reduce image feature amount, and it is possible to utilize a “convolution type neural network” that operates with minimal processing and has strong pattern recognition. It is also possible to utilize a “recursive neural network” (fully connected recurrent neural network) that handles more complicated information, and with which information flows bidirectionally in response to information analysis that changes implication depending on order and sequence.

In order to realize these techniques, it is possible to use conventional general purpose computational processing circuits, such as a CPU or FPGA (Field Programmable Gate Array). However, this is not limiting, and since a lot of processing of a neural network is matrix multiplication, it is also possible to use a processor called a GPU (Graphic Processing Unit) or a Tensor Processing Unit (TPU) that are specific to matrix calculations. In recent years a “neural network processing unit (NPU)” for this type of artificial intelligence (AI) dedicated hardware has been designed to be capable being integrally incorporated together with other circuits such as a CPU, and there are also cases where they constitute some parts of processing circuits.

Besides this, as methods for machine learning there are, for example, methods called support vector machines, and support vector regression. Learning here is also to calculate discrimination circuit weights, filter coefficients, and offsets, and besides this, is also a method that uses logistic regression processing. In a case where something is determined in a machine, it is necessary for a human being to teach the machine how determination is made. With this embodiment, determination of an image adopts a method of performing calculation using machine learning, and besides this may also use a rule-based method that accommodates rules that a human being has experimentally and heuristically acquired. Still other methods of machine learning that may be used include Bayesian networks.

In this way, an inference model is generated in the customized learning section 33, and if this inference model is set in the inference engine 11 it becomes possible to provide various dietary advice to the user. For example, when the user 90 has visited a convenience store 41, restaurant 43, or food delivery center 45, etc., holding the information terminal 10, if images of ingredients and dishes are acquired it is possible to provide advice relating to diet to the user 90.

The convenience store 41, restaurant 43, and food delivery center 45, etc., respectively, often have computers such as servers, etc., for merchandise control (called store servers), and it is possible to perform control in association with the computer 30 and information terminal 10. Computers such as these store servers operate by cooperating while sharing exchange, retrieval, and display control, etc., of data, making it possible to provide the services such as in this application to the user. These computers cooperate with databases, etc., provided for the product management, ordering management, and delivery management described here, and also form a network that is appropriate to other stores and related organizations, and enabling collaboration.

For example, as shown in FIG. 12, if an image is acquired by pointing a camera at ingredients and dishes, a computer of this information terminal, or a computer on the cloud, etc., that cooperates with the information terminal, determines at what positions what dishes and food are within a screen, and classifies the screen for every dish constituting an object of determination. The computer then infers ingredients and cooking method for every position within the screen that has been respectively classified, based on characteristics of that dish (including desserts and beverages), and for every ingredient, and material and cooking method, that has been obtained with that inference result, searches for what type of effect there will be on the human body (in the case of pets or animals, the type of effect on respective animals) using a database or the like, and determines so as to output “OK” if ingredients and cooking method are acceptable. By performing this processing, it is possible for the user to be able to easily confirm in which region(s) within a screen there are food and/or beverage item(s) that can be ingested. At this time, if it is made possible to determine size (weight) of dishes based on images, and made possible for the user to input rough estimates and measurement values, it is possible to also give advice regarding dietary intake by performing comparison and inference, etc., with ideal dietary intake in a database. Naturally, it is possible to input user height and body weight information, and to take this information into consideration.

A computer of the information terminal, or a computer on the cloud etc. that cooperates with the information terminal, takes account of user status, and for dishes within a screen, displays ◯ in the case of food that is good to eat and displays X in the case of food that should not be eaten (refer to FIG. 12). Since there are items that cannot be made out because an image is indistinct, or too small, or shaded, in this case AI reliability is set low, and “?” etc. shows that it cannot be determined from the given image whether or not the food can be eaten, or improved imaging should be obtained. Also, in a case where the user 90 has visited an online net supermarket (also referred to as a net supermarket) that is capable of home delivery of ingredients, if images of ingredients and dishes are selected, then similarly display of ◯ and X is performed. Similar determination is also possible with images on a network. It should be noted that images that have been made into this type of electronic data are input directly to an inference engine without being photographed, and the above described determination for ◯ and X is performed, and may be notified to the user. Without being limited to performing determination for ◯ and X, favorability may be shown using some numerical value, may be represented visually using color or marks, or framed display etc., or may be represented using characters or voice, etc.

Also, the computer 30 acquires dietary habit needs of users without limiting to specified people, and if it is possible to acquire information such as that there are people affected by similar health problems, it is possible to advise those people that it is possible to provide food that is good for health. Specifically, it is possible to provide information that is useful when examining ingredients and diet, and it is possible to advertise products for convenience stores, etc. Also, it is possible to provide information in the reverse direction; that is to restaurants, grocery stores, food delivery services, etc. For example, if there is a large population of users that require certain food or beverage items, or that would be good potential customers for certain food or beverage items, grocery stores, food delivery services, etc. could be advised to stock or offer such items, especially if such items are not widely available.

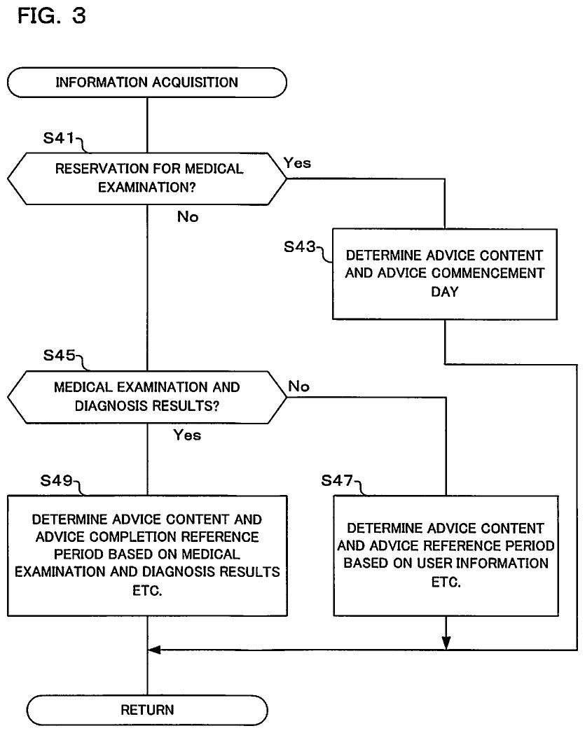

Next, operation of a chatbot of the information terminal 10 within a user auxiliary information output system will be described using the flowcharts shown in FIG. 2A and FIG. 2B. This flow is mainly executed by the CPU within the control section 11 controlling the information terminal 10 in accordance with programs that have been stored in memory (it should be noted that this also applies to the flow shown in FIG. 3 to FIG. 7B).

If the flow for the chatbot shown in FIG. 2A is commenced, it is first determined whether or not to acquire medical examination and consultation information (S1). At the time that a user makes a reservation or request for a medical examination, consultation, procedure, etc., at a medical facility or examination agency such as the municipal hospital A 20 or district hospital B 25, there are cases where it is possible to request receipt of notification of examination and consultation information. For example, in a case where a user will register at reception of a medical facility or examination agency, that medical facility may automatically transmit examination and consultation information, and by installing application software for information receipt on the information terminal 10 the medical facility may automatically transmit the examination and consultation information. A code (for example a QR code (registered trademark)) for application software input, etc., is prepared at reception, and it may be possible for the user to easily install the application software. It should be noted that the application software may be installed at the time of requesting examination, after examination, or after examination results have been heard or otherwise received, etc. Also, if the application software is installed, advice may be output until the next examination, etc., and it is possible to improve symptoms, etc.

In a case where setting has been performed such that the medical facility automatically transmits medical consultation, examination, and/or procedure information, if the medical facility performs medical consultation, examination, and/or procedure for the user then the results of that medical consultation, examination, and/or procedure will be transmitted to the information terminal 10 as diagnosis and examination information 72, and in this step S1 it is determined whether or not the information terminal 10 has acquired this information. Also, without being limited to the transmission of consultation or examination results, when the user has made a reservation for a medical consultation, examination, and/or procedure, this fact can be acquired as consultation and examination information 72.

If the result of determination in step S1 is that it was determined that medical consultation, examination, and/or procedure information will be acquired, that medical consultation, examination, and/or procedure information is acquired (S3). Here, the control section 12 determines whether or not examination or the like has been booked, and whether or not medical consultation, examination, and/or procedure has been received, and determines whether or not a medical facility, such as the municipal hospital A 20 or the district hospital B 25, has transmitted diagnosis and examination information 72 to the information terminal 10 as information relating to reservation, medical consultation, examination, and/or procedure. If the result of this determination is that diagnosis and examination information 72 has been received, the information terminal 10 inputs the diagnosis and examination information 72 and stores in the storage section 14.

In information acquisition, if endoscopic examination has been performed in the municipal hospital A 20 or district hospital B 25, resulting endoscopic images may be acquired. The medical examination and consultation information that was acquired in step S3 may be stored as diagnosis results for the user. Also, without being limited to endoscope images, the medical facility, etc., may input images that have been acquired at the time of diagnosis or examination such as x-ray images, MRI (Magnetic Resonance Imaging) images, CT (Computed Tomography) images, etc., and store these images that have been input. Detailed operation of the information acquisition of step S3 will be described later using FIG. 3.

If processing for information acquisition has been performed in step S3, or if the result of determination in step S1 is that medical examination and consultation information is not required, next, the day-to-day status of the user is determined (S5). Here, the control section 12 collects information relating to day-to-day status of the user, and determines day-to-day status of the user based on this information. This information relating to day-to-day status may be, for example, user status information 71 from the municipal hospital A 20, etc., information entered by the user via operation section 16, information that has been acquired by the sensor section 17 of the information terminal 10, user mail information, information that has been contributed to SNS (e.g., user upload of photos, diary entries, etc., about meals) etc., and user information that can be collected by the information terminal 10. Information may also be collected in cooperation with smart appliances that are utilized by the user 90.

Also, if position of the information terminal 10 is known from GPS, etc., within the sensor section 17, it is possible to obtain movement information of the user in possession of the information terminal 10, for example, information such as performing an activity such as running, etc., as status information of the user. Also, the control section 12 may collect vital information such as the user's sleep time, mealtimes, number of steps, amount of exercise, body temperature, blood pressure, pulse rate, blood oxygenation level, weight, body fat percentage, blood glucose level, toilet information, etc., using the built in sensor section 17 or using a sensor section of a portable unit (in this case also, it can be considered that the sensor section 17 is separate). Without being limited to only these types numerical values and data, it is also possible to make use of variation in this data over time in making determinations of user status information. In this way it is possible to adapt to suit changes in body condition. Further, if the sensor section 17 of the information terminal 10 has a photographing section, day-to-day status determination may also be performed using photographs of the users themselves, for example, photographs of meals the user has eaten.

Further, status information of the user may be, for example, time from when the user underwent diagnosis, current time, profile of the user (gender, age, etc.), user lifestyle habits, user medical history, user ingestion history, etc. As lifestyle habit information of the user, for example, times of meals, eating habits, smoking, drinking, and overeating and over-drinking tendencies, exercise, content and style of work, sleep tendencies, and expressions, and change tendencies for face color, etc., from facial photographs etc., constitute useful information. Also, if medical history information of the user is known, then in cases of regurgitant gastroenteritis, for which it is necessary to change from normal advice, with allergies, etc., various treatments are known, such as it being necessary to use, as well as ingredients and cooking methods, meals for which reflux is unlikely to occur. Also, as ingestion history of the user, there are, for example, dosages of tranquilizers, and drugs to improve blood flow, etc.

Next, it is determined whether or not information on consultation is necessary (S7). Here, the control section 12 determines whether or not information on consultation and examination etc. is necessary, in order to provide advice to the user. There are cases where results of consultation and examination are relevant when providing information to the user (refer to S5), based on results of the control section 12 having determined medical examination and consultation information and day-to-day information of the user. On the other hand, there are cases where it is possible to provide advice to the user even if there is no information on consultation time. For example, in a case where eating something sweet before going to sleep is detrimental to health, or for inactive users, in a case of promoting activity, etc., it is possible to provide advice to the user even if there is no information for at the time of consultation. With this step, therefore, it is determined whether or not information that has been acquired at the time of consultation or examination is required in order to provide advice to the user.

If the result of determination in step S7 is that it is been determined that information at the time of consultation is not required, normal advice is performed (S9). In this case, normal advice is performed without information at the time of consultation being necessary. For example, in a case where a user is living an irregular lifestyle, the control section 12 provides irregular lifestyle measures as advice. Also, the control section 12 provides advice to the user that does not require information at the time of consultation, such as the pros and cons of bathing and drinking alcohol, based on day-to-day information. Advice relating to diet that does not require information at the time of consultation is performed in this embodiment in steps S13 and S15, which will be described later.

If advice is output in step S9, or if the result of determination in step S7 is that information at the time of consultation is required, it is next determined whether or not there is information that depends on time (S11). In a case where information that is related to diet is provided to the user, there are cases where dietary advice changes in accordance with time from the time of consultation, the time of examination, the time of the procedure, etc. For example, there are cases where diet is restricted after an examination, and in this case also the content of dietary restriction changes as time goes by. Before an examination also, content of dietary restriction changes with time. In a case of having undergone surgery at a medical facility or the like also, dietary restriction similarly changes with time. Further, without being limited to dietary restriction, there are cases where meal content (menus) that is recommended also changes with time. In this step, in a case where dietary advice is given in accordance with results of consultation or examination of the user, or day-to-day status of the user, the control section 12 determines whether or not to change that dietary advice over time.

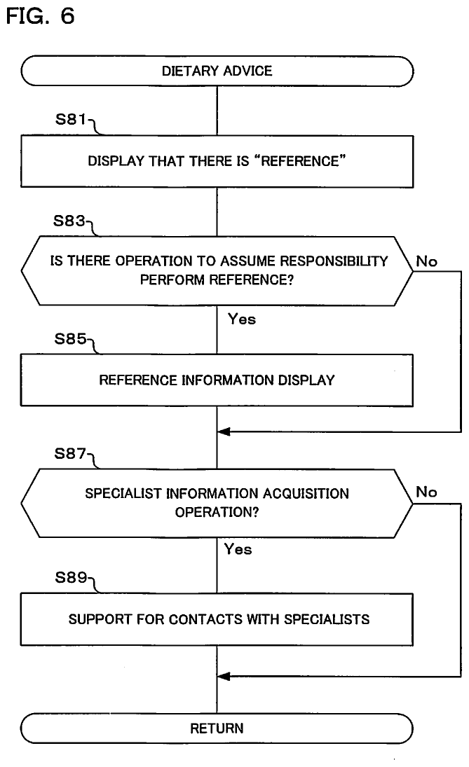

If the result of determination in step S11 is information that is not dependent on time, normal dietary advice is given (S15). In this case, the control section 12 performs dietary advice that can be implemented without considering chronological change, such as, for example, countermeasures for allergies, chronic diseases and diseases related to lifestyle habits (including overeating and over-drinking measures). This dietary advice is presented to the user on the display section 15 of the information terminal 10, but other methods of presentation may also be used. Detailed operation of this normal dietary advice will be described later using FIG. 5. Also, when performing this normal dietary advice, advice that is given in a supplementary manner will be described later using FIG. 6.

On the other hand, if the result of determination in step S11 is information that is dependent on time, dietary advice that depends on time is given (S13). Here, the control section 12 performs dietary advice taking into consideration chronological change from the time of consultation, etc. For example, dietary advice is given taking into consideration the name of a disease that the user is affected by, and the degree of symptoms, etc. Dietary advice is also changed based on images that were acquired at the time of consultation and/or examination. For example, in a case where position and size of polyps that have been excised is known based on endoscopic images, how to provide meals may be determined and advice output based on these images. There will sometimes be cases where tumors and lesions other than polyps are excised, but here the “polyp” is used as one specific example. Also, dietary advice is not restricted to after consultation and after examination, and may also be given in accordance with dietary restrictions before consultation and before examination. Dietary advice is presented to the user on the display section 15 of the information terminal 10, but other methods of presentation may also be used. Detailed operation of this dietary advice will be described later using FIG. 5. Also, when performing this time dependent dietary advice, advice that is given in a supplementary manner will be described later using FIG. 6.

If dietary advice has been performed in steps S13 or S15, it is next determined whether or not negotiation has occurred (S17). Users that have been provided with dietary advice have various circumstances, and may oppose the advice that has been presented, with the result that advice is not followed. Also, there are cases, when following advice, where assistance using the information terminal 10 is not necessary. In this type of case, the fact that assistance is not necessary or is ignored by the user is input to the information terminal 10 by means of the operation section 16 etc. If the result of determination in step S17 is that the user has provided such an input, it is determined that negotiation has occurred. If the result of determination in step S17 is that negotiation has not occurred, processing returns to step S1.

If the result of determination in step S17 is that negotiation has occurred, it is next determined whether or not there is supplementary help (S21). In a case where negotiation is taking place, there are cases where detailed information is required in order for the user to follow dietary advice, and cases where customized information is required. In these cases, the user inputs the fact that supplementary help is required by means of the operation section 16 of the information terminal 10, and the control section 12 performs determination based on whether or not this input has been supplied. It should be noted that in the case where the user requires customized information, depending on the content of customized information it may be provided on the assumption that it will be charged for.

If the result of determination in step S21 is that there is no supplementary help, alternative advice is provided in response to negotiation (S23). When providing dietary advice, the control section 12 may prepare numerous items of advice, and may assign order of priority among the plurality of items of advice. In a case where there is negotiation and supplementary help is not required (a case of S17=Yes->S21=No), alternative advice is provided sequentially from among the plurality of items of advice. Specifically, when the dietary advice has been provided to the user in steps S13 and S15, if the user refuses this advice alternative proposals are sequentially presented.

If the result of determination in step S21 is that supplementary help is required, corresponding aids are retrieved (S25). Even if the user follows the dietary advice that was presented steps S13 or S15, there are cases where the advice cannot be implemented immediately. For example, when ingredients or dietary content that is required for the dietary advice part has been presented in steps S13 or S15 is not at hand for the user, if they do not make purchases at shops that are nearby, or make use of services, etc., it is not possible to follow the dietary advice. If this type of situation is input to the information terminal 10 by the user using supplementary help, the control section 12 retrieves means of assistance corresponding to this supplementary help (for example, nearby stores and services that can provide).

Also, in a case where “food that is good for indigestion” has been recommended as dietary advice, there are cases where, as a user, it is not known what ingredients and meals are good for indigestion. In this type of case therefore, examples of ingredients and meals that are good for indigestion may be presented, and shops and services that can provide ingredients and/and meals that are good for indigestion may be presented.

Next, it is determined whether or not corresponding means of assistance can currently be provided (S27). Here, the control section 12 determines whether or not it is currently possible to provide means of assistance to the user based on corresponding means of assistance that have been retrieved in step S25. For example, it is determined whether or not ingredients and meal menus that are necessary in order to execute the dietary advice that has been presented in steps S13 and S15 can be currently supplied, from among shops and delivery services, etc., that are capable of supplying these items to the user within a predetermined time period.

If the result of determination in step S27 is that ingredients and menu items can currently be supplied, providable information is displayed (S29). Here, the control section 12 displays providable information based on results that were determined in step S27. When providing this information, service information such as shop information, or delivery services, or restaurants, that are capable of providing meals in accordance with ingredients and/or cooking methods may also be included (Refer, for example, to S93 in FIG. 7A, and S103 in FIG. 7B). Also, information such as which convenience stores are good, and which delivery services are good, etc., may also be provided. Detailed operation of this providable information display will be described later using FIG. 7A and FIG. 7B.

If the result of determination in step S27 is that items cannot currently be provided, it is determined whether or not reservation is possible (S31). There are cases where it is possible to procure meals and ingredients by means of reservation, even if they cannot currently be supplied (or cannot be supplied or obtained within a predetermined time period) by shops and delivery services, etc. In this step therefore, if there are shops, etc., where reservation is possible, this fact is provided to the user by the control section 12.

If the result of determination in step S31 is that reservation is possible, a reservation procedure, etc., is presented (S33). Here, the control section 12 presents shops and services that are capable of reservation to the user, and provides assistance so the user can undertake the reservation procedure. In this case, a plurality of shops and services are displayed, the user selects a shop etc., and the reservation may be performed by means of the information terminal 10.

If the reservation procedure is performed in step S33, or if the result of determination in step S31 is that reservation is not possible, or if display of providable information is performed in step S29, or if alternative device is provided in step S23 in accordance with negotiation, processing returns to S17.

In this way, in the chatbot flow shown in FIG. 2A and FIG. 2B, user status information is input (refer to S5), diagnosis information relating to diagnosis of the user, etc., are provided as inputs (refer to S3), and advice relating to user diet is output based on information relating to diagnosis and user status information (refer to S13 and S15). Advice relating to diet may also be provided as information that is a combination of ingredients and cooking methods.

Also, advice that has been output by the advice output section should be approved by a doctor, nutritionist, or health care professional before the user takes meals. For example, a step may be added, before or after dietary advice is presented in steps S13 and S15, for approval by a doctor or the like to be requested. Also, in steps S13 and S15, etc., advice may be performed given on images that have been obtained at the time of diagnosis. For example, in a case where endoscopic images are required at the time of diagnosis, advice may be output in accordance with size of polyps that has been obtained from the endoscopic images.

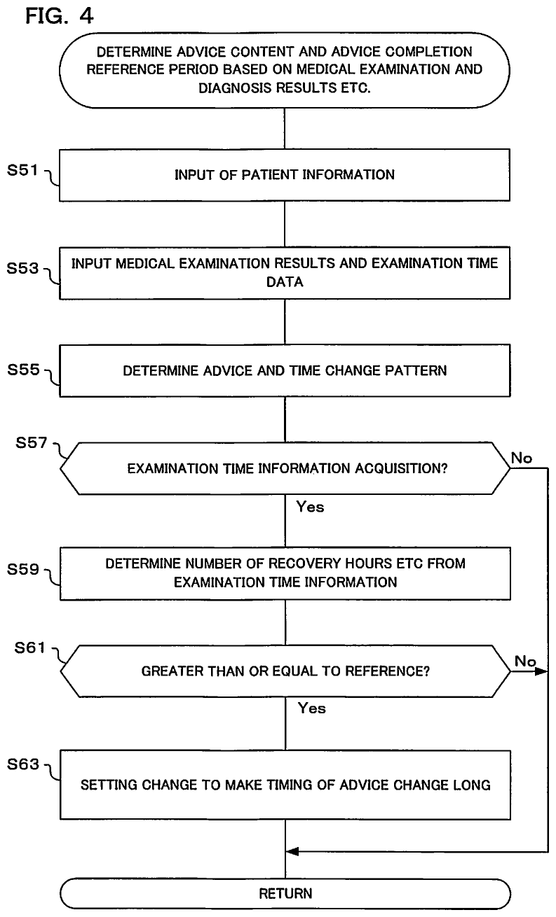

Also, a period in which advice is performed may be changed based on images that have been obtained at the time of diagnosis. For example, content of advice relating to diet, or a period in which advice relating to diet is output, may be changed in accordance with the size of polyps at the time of diagnosis. Generally, in a case where it has been identified from an image that symptoms are bad, since it will take time for a complete recovery, a period for which advises output is made long. However, in a case where symptoms have eased from normal, from a safety viewpoint a period in which advice is output may be set to a standard period (refer to S61 and S63 in FIG. 4).

In this way, as a user auxiliary information output of this embodiment, in a case where this information terminal 10 has been assumed, the control section 12 may assume control, and control may be assumed by cooperation with a computer 10 on the cloud (in particular, the control section 35). At this time, it is assumed that there is a configuration having a user status information input section for inputting status information of the user (here, this is assumed to be, for example, information that results from having stored information of the sensor section 17 in the storage section 14, results acquired from in-hospital systems and medical appliances by means of the communication section 13, or information that has been stored in the storage section 14), and an advice output section for outputting advice relating to shops or services capable of supplying meals that are suitable for the user, in accordance with the status information, by means of coordination of information possessed by POS systems of the shops or services (here, the control section 35, for example).

It should be noted that with this embodiment, advice has been provided to the person who has the information terminal 10 (refer, for example, to S9, S13, and S15 in FIG. 2A, and to S23, S29 and S33 in FIG. 2B). However, since there are also cases where a person making meals is not the person in question but their spouse, partner, or housemate, the advice may also be provided to these people. It can also be envisaged that the person's spouse, etc., may go shopping.

Also, in this embodiment, if drugs that have been prescribed at the hospital, etc., or over-the-counter drugs that have been purchased at drugstores, are also stored in a database within the storage section 14 (or alternatively a storage section within the computer 30) as diagnosis and examination information and status information (refer for example to steps S3 and S5), a relationship between drugs, etc., and meals can also be ascertained. It is possible to provide dietary advice to the user based on these items of information (refer, for example, to steps S13 and S15). It becomes possible to monitor the health of the user by means of cooperation between shops, and between services.

Next, operation for information acquisition (refer to S3 in FIG. 2A) will be described using the flowchart shown in FIG. 3. As was described previously, in the information acquisition of step S3, the control section 12 acquires diagnosis and examination information 72 from medical facilities and examination agencies, etc., and performs various determinations.

If the flow for information acquisition shown in FIG. 3 is commenced, it is first determined whether or not there is a reservation for a medical examination (S41). Here, the control section 12 determines whether or not the diagnosis and examination information 72 that has been acquired is information relating to a reservation for a medical examination. It should be noted that before execution of step S41, in the event that patient information is not clear, this patient information may be acquired first. Patient information is input based on information that has been transmitted from medical facilities, etc., such as the municipal hospital A 20 and district hospital B 25 including diagnosis and examination information 72. Information with which it is possible to identify a patient, such as name, is preferable as patient information, but various information may also be provided in addition, such as gender, age, and profile, etc.

If the result of determination in step S41 is that the information that has been acquired is a reservation for a health examination or procedure, advice content and the day for advice commencement are determined (S43). There are cases where dietary restrictions are in place before examination, and further there are cases where fasting must be performed for a specified period of time, and there is dietary content recommended in accordance with examination. The control section 12 therefore determines content of advice and advice commencement date based on patient information, healthcare examination content, type of medical examination or procedure, and/or healthcare examination reservation date, etc.

If the result of determination in step S41 is that a healthcare examination or procedure has not been reserved, it is next determined whether or not information that has been acquired is healthcare examination, procedure, and/or diagnosis results (S45). Here, the control section 12 acquires diagnosis and examination or procedure information 72, and determines whether or not the diagnosis and examination or procedure information 72 that has been acquired is information relating to results of healthcare examination or diagnosis.

If the result of determination in step S45 is that the information that has been acquired is not healthcare examination, procedure, or diagnosis results, then advice content and advice reference period are determined based on user information, etc. (S47). Here, the control section 12 determines advice content and reference period for providing advice based on user behavior information and vital data, etc., of the user. Specifically, since the information that has been acquired is not health examination results, medical procedure results, or diagnosis results, etc., the control section 12 determines content of dietary advice that will be provided to the user, and criteria for the period in which this advice will be provided, based on history information that has been acquired in relation to the user up to now, for example, user information, user behavior information, and vital information of the user, etc.

On the other hand, if the result of determination in step S45 is that the information that has been acquired is healthcare examination, healthcare procedure, or diagnosis results, then advice content and advice completion reference period are determined based on healthcare and diagnosis results, etc. (S49). Since the information that has been acquired is healthcare and diagnosis results, here, the control section 12 determines advice content, and a reference period for a period in which advice will be completed, based on patient information, healthcare results and/or examination data (including medical appliance information such as wound images at the time of treatment) etc. Since the information that has been acquired is healthcare examination results, healthcare procedure results, and/or diagnosis results, these items of information are included, and the control section 12 determines content of dietary advice provided to the user, and criteria for a period in which this advice will be completed. Detailed operation in this step will be described later using FIG. 4.

If the processing of steps S47, S49 or S43 has been executed, the flow for this information acquisition is terminated and the originating flow is returned to.

Next, determination of advice content, and advice completion reference period, based on healthcare examination, healthcare procedure, and/or diagnosis results (refer to S49 in FIG. 3) will be described using the flowchart shown in FIG. 4. As was described previously, in step S43 the control section 12 determines advice content and advice completion reference period by reflecting medical appliance information, using healthcare and diagnosis results, etc.

If the flow shown in FIG. 4 is commenced, first, patient information is input (S51). Patient information is input based on information that has been transmitted from the municipal hospital A 20 and district hospital B25, including diagnosis and examination information 72. If patient information is input before execution of step S41, this step may be omitted.

Next, physical examination results and examination time data are input (S53). The control section 12 inputs healthcare examination results and examination time data based on diagnosis and examination information 72 from the municipal hospital A2 0 and district hospital B 25.

Next, advice and time change patterns are determined (S55). Dietary advice will be provided after it is determined in accordance with healthcare examination results, healthcare procedure results, etc. As time passes from the medical examination or procedure time, dietary restrictions are gradually relaxed. The dietary advice is therefore changed in accordance with elapsed time from this medical examination or procedure time. In this step the control section 12 determines changes in the user's eating patterns over time (for example, at time T1 of the first day user eats bread with butter and eggs as breakfast, and at time T2 of the first day he/she eats rice and soup as lunch, etc.) for the dietary advice that will be provided.

It is next determined whether or not to acquire examination or procedure time information (S57). Depending on status at the time of examination, for example, depending on status of wounds at the time of treatment, time until various milestones in recovery will be different. Therefore, in order to estimate recovery time(s), the control section 12 performs determination as to whether or not examination time information is required.

If the result of determination in step S57 is that it is necessary to acquire medical examination or procedure time information, a number of recovery hours, etc., is determined from the time of the medical examination and procedure time (S59). Here, the control section 12 determines a number of days, etc., required until recovery (number of recovery hours, etc.) based on the examination or procedure time information that has been acquired. For example, in a case where wound images have been acquired as examination time information, the control section 12 determines locations and range of damage, and calculates a number of recovery hours from this information.

Next, it is determined whether or not the number of recovery hours, etc., that has been determined is greater than or equal to normal (S61). As was described previously, dietary advice is performed from examination or procedure time, but this dietary advice changes with passage of time, in accordance with examination or procedure time information, finally resulting in a “normal” diet. Here, the control section 12 determines whether or not a number of days (number of recovery hours, etc.) until a return to a normal diet is realized is longer than a number of days (normal number of days) that is presumed to be normal. Specifically, since, depending on condition at the time of examination, there are cases where it takes a greater number of days for the user to recover than a normal number of days (refer to S57 and S59), in this step this is determined. If the result of determination in step S61 is not greater than normal, then it might be good to shorten the number of days until the dietary advice becomes a normal condition, but here, dietary advice is performed for the normal number of days, from the viewpoint of safety.

If the result of determination in step S61 is greater than normal, setting is changed so as to make time for advice change longer (S63). Since the number of recovery hours that was obtained in step S59 is longer than normal, the control section 12 performs setting change so at to make a time for changing dietary advice longer.

If the result of determination in step S57 is that examination or procedure time information is not required, or if the result of determination in step S61 is that the number of recovery hours, etc., is not greater than normal, or if the processing of step S63 is executed, the flow shown in FIG. 4 is terminated and the originating flow is returned to.

Next, dietary advice (refer to S13 and S15 in FIG. 2A) will be described using the flowchart shown in FIG. 5. As was described previously, in steps S13 and S15 the control section 12 determines content of dietary advice provided to the user, and displays dietary advice on the basis of this determination. In this flow, advice regarding whether ingredients and meals are suitable is presented in accordance with user symptoms.

If the flow shown in FIG. 5 is commenced, first, recommended ingredients are determined (S71). Here, the control section 12 determines ingredients that have been recommended to be eaten by the user based on medical examination and consultation information (refer to S1 and S3 in FIG. 2A) and day-to-day information of the user (refer to S5 in FIG. 2A). It should be noted that in the case of the dietary advice of step S13, since recommended ingredients will differ with elapsed time from the time of consultation, etc., determination of whether or not there are recommended ingredients takes this time into consideration. Alternatively, or in addition, ingredients to be avoided may be considered.

Next, a recommended food preparation (e.g., cooking) method is determined (S73). Here, the control section 12 determines meals to be eaten by the user based on medical examination, procedure, and/or consultation information (refer to S1 and S3 in FIG. 2A) and day-to-day information of the user (refer to S5 in FIG. 2A). It should be noted that in the case of the dietary advice of step S13, since recommended ingredients will differ with elapsed time from the time of consultation, etc., determination of whether or not there is a recommended cooking method takes this time into consideration.

Next, dishes that satisfy conditions of recommended ingredients and recommended food preparation (e.g., cooking) methods are determined (S75). Here it is determined whether or not there is at least one cuisine or meal that satisfies the recommended ingredients and recommended cooking method that were determined in steps S71 and S73. It should be noted that an inference model for determining recommended ingredients and recommended cooking method may be set in the inference engine 11, and that the determination in steps S71 and S73 may be performed based on these inference results. Alternatively, a database may be constructed in the storage section 14 of the information terminal 10, and the determination in steps S71 and S73 may also be performed by referencing this database. Dishes that meet the conditions of step S75 (this may also be ingredients only) are presented to the user.

It is next determined whether or not there is image reference (S77). For example, there are cases where it is desired to know whether or not dishes that it appears the user will eat from now on conform with the dietary advice, as shown in FIG. 12. In this case, it is useful to display determination results directly on the display section 15 (and in some embodiments, directly over each meal or item of food included in a meal) of the information terminal 10. Therefore, with this embodiment, the imaging section within the sensor section 17 of the information terminal 10 acquires images of food, these images are identified, and it is determined whether or not the conditions of step S75 are satisfied based on the results of this identification. In this step, the information terminal 10 acquires images, and the user determines whether or not they want the dietary advice on the basis of these images. It should be noted that even if an imaging section is not provided, images of food may be input and this type of determination may be performed.

If the result of determination in step S77 is that there are reference images, those that satisfy conditions and those that do not satisfy conditions are determined, and one or more indications of a satisfied condition and/or an unsatisfied condition is displayed (S79). Here, the control section 12 analyzes images that have been acquired by the imaging section, and determines what ingredients there are, and what cooking methods are used. It is then determined whether or not ingredients and food preparation (e.g., cooking) methods that have been determined satisfy the conditions that were obtained in step S75, and the determination results are displayed (refer to FIG. 12).

If the display in step S79 has been executed, or if the result of determination in step S77 is that images are not referenced, the flow for dietary advice is terminated and the originating flow is returned to.

With this flow for dietary advice, it is possible to perform appropriate dietary advice at places other than at hospitals and nursing facilities, in accordance with user symptoms. For example, in the case of a user suffering from dyslipidemia, there will be menus that are good to eat during the course of their day-to-day life, but it will often be the case that they will not know what ingredients and food preparation (e.g., cooking) methods are not suitable for their condition. There is LDL cholesterol, HDL cholesterol, and triglyceride (neutral fat) within lipids in blood, and dyslipidemia generally refers to a condition where, in the lipids within blood, there is high LDL cholesterol, low HDL cholesterol, and/or high triglyceride. If a condition where a value for LDL cholesterol is high continues, arteriosclerosis will advance further. On the other hand, if excess HDL cholesterol that has built up on blood vessel walls is removed it will slow or halt the advancement of arteriosclerosis. Also, if a condition of high triglyceride continues, then the risk of myocardial infarction, angina angiitis, and cerebral infarction, etc., is increased. In addition to dyslipidemia, with metabolic syndrome where conditions of high blood glucose level and blood pressure combine, the risk of arteriosclerotic diseases such as myocardial infarction is further increased. In this type of case where a patient has been affected by dyslipidemia, if certain types of ingredients are not suitable and certain types of food preparation (e.g., cooking) methods are not suitable, these contraindications are displayed, it is extremely beneficial to the user.

When providing dietary advice, information on calories, salt content, carbohydrate, fat, and protein may also be used. Protein is important to human health, but if too much protein is ingested, the workload on the kidneys becomes large. Also, primarily, users who are suffering from health problems such as heart disease, kidney disease, high blood pressure, and hypertension due to pregnancy should restrict their salt intake (e.g., to less than 6 g per day). If the daily sodium intake limit is 6g, sodium content contained in ingredients for one day (perishables, etc.) is considered to be 1g, salt content that can be added to these meals becomes 5 g.

Also, at the time of determination in steps S71 and S73, the database such as shown in FIG. 9 may be searched, and an inference model that was created by the customized learning section 33 utilizing machine learning may also be used. Here, similarly to FIG. 1C, a conceptual example that is easy to understand has been taken, but a database may be created in which image information, and material information and food preparation (e.g., cooking) information, etc., may be collectively stored. The care food data sets 1 to 3 that were shown in FIG. 1A to FIG. 1C may also be used as training data for creating an inference model. Since care food can be put on menus that have been confirmed as suitable for symptoms of various patients by nutritionists and the like, it becomes possible to perform appropriate dietary advice by referencing these care food data sets 1 to 3. Also, images of food that have been published on food sites, etc., on the Internet may be made into training images, and machine learning performed. Further, ingredients and ingredient amounts of approved care food may be determined, and other dishes with these ingredients and amounts can be searched for.

In FIG. 9, relationships between affected parts and diet are made into a database. However, if relationships between affected parts and symptoms, treatment status, healing progress information, ingredients, and food preparation (e.g., cooking) methods is arranged in this database, then it becomes possible to separate into materials, cooking methods, and processing methods, and to determine respective suitability, even in cases where new food appears. Also, if image data, and information on the fact that that alone is insufficient, are also arranged in this database, as was described previously, it is possible to display shortage and surplus information collectively, regardless of whether that food is appropriate for ingestion or not

If training data is created by arbitrarily processing the database that has been arranged in this way and an inference model has been learned using this training data, combined use with AI technology may be performed. For example, in cases such as when making judgment based only on appearance, because image inference technology has advanced it is possible to utilize suitability for ingestion using images. The database here in which ingredients and food preparation (e.g., cooking) methods have been combined preferably further includes image information for food and/or ingredients, and food name information, and material name information, in order to be able to retrieve those food names using images. This is in order to make this kind of information that has been made into text easier to search more effectively etc.

Also, a database in which ingredients and food preparation (e.g., cooking) methods have been combined may also further include temperature information at the time of serving food, the reason for this being that, depending on body condition and body composition, there are effects on health due to temperature, such as due to cold items. Naturally temperature information for the time of processing is also important for infection control measures, etc. The database in which ingredients and food preparation (e.g., cooking) methods have been combined here preferably further includes surplus and shortage and/or quantity information for materials or cooking methods, and as a result it becomes possible to give advice to the user regarding insufficient nutrients and calories with only that food. Also, since calories change with dietary intake, as well as the size and weight of food, information on amount or weight with reference to bowls and plates is also stored, or determination may be made possible. In this case, it may be made possible to determine weights from images.

Also, in step S79, advice for displaying suitability of the user eating something is output for every position of individual food items within an image (refer to FIG. 12). Alternatively, only foods satisfying conditions, or only foods failing to satisfy conditions, might be indicated with a visual cue on the display. In this case, instead of just simple display, advice that has been output, or information that was used when creating advice, may be shared with facilities (may also be the computer 30, etc.) that cooperate with the information terminal 10 (may also be, for example, medical facilities or examination agencies such as the municipal hospital A 20 and district hospital B2 5), or a specialist, etc. By sharing this information, it also becomes possible to obtain advice for ingredients and drinks that can be ingested etc. from relevant agencies.

Also, in steps S77 and S79, since taking pictures of food is performed before meals, medicine that should be taken before meals may be instructed to the user using this picture caking timing. Medicine that should be taken after meals may also be instructed to the user at a time after the meal, based on status information, etc. (refer to step S5). This case is not limited to displaying information on the display section 15 of the information terminal 10, and the user may also be notified by means of voice or superimposed display, or icon display for a separate screen, etc. By presenting these items of advice, it is possible to simply output advice to remember to take medicine and to avoid mistakes.

Next, supplementary dietary advice that is performed for the dietary advice (refer to S13 and S15 in FIG. 2A) will be described using the flowchart shown in FIG. 6. As was described previously, in steps S13 and S15 the control section 12 determines content of dietary advice provided to the user, and displays dietary advice on the basis of this determination. The flow shown in FIG. 6 may be executed before the flow for dietary advice that was shown in FIG. 5, and in a case where the user has consented to there being reference information in the dietary advice (refer to S83) the dietary advice shown in FIG. 5 may be presented (refer to S85).

If the flow shown in FIG. 6 is commenced, first the fact that there is “reference” dietary advice for the user's consideration is displayed (S81). There are cases where, if dietary advice such as shown in steps S13 and S15 is displayed on the information terminal 10, the user thinks that they absolutely have to implement that advice. However, the dietary advice has reference information, and ultimately diet content is something that is determined by the user. Therefore, the control section 12 notifies the user of the fact that the dietary advice has reference information.

Next, it is determined whether or not an operation, indicating that the user has understood that referring to this information is their own responsibility, has been performed (S83). At the time of displaying dietary advice on the information terminal 10, icons for performing gesture display to accept that referring to the dietary advice is the user's own responsibility are displayed on the display section 15. In this step, the control section 12 determines whether or not intention has been displayed by the user performing an operation, such as clicking on this icon. It should be noted that this operation may also be performed by operation of an operation member such as a button, as well as operation of an icon.

If the result of determination in step S83 is that an operation to refer to the dietary information on their own responsibility has been performed, reference information is displayed (S85). Here, dietary advice is displayed as reference information, in accordance with the flow such as shown in FIG. 5, for example.

If the reference information has been displayed in step S85, or if the result of determination in step S83 is that an operation to accept responsibility for reference has not been performed, it is next determined whether or not an operation for specialist information acquisition has been performed (S87). In cases where the user conforms to dietary advice as reference information, and in cases where they do not, specialist recommendations are very useful. In this step, therefore, it is determined whether or not the user has performed an operation in order to acquire specialist recommendations.

If the result of determination in step S87 is that an operation has been performed in order to acquire specialist information, contact with the specialist is supported (S89). Here, for example, the information terminal 10 may display a list of specialists, or contact information of specialists, etc., for the purpose of contact with specialists. For this purpose, enterprises that operate the chatbots using the information terminal 10 may build a network with specialists.

Once contact with specialists has been supported in step S89, or if the result of determination in step S87 was that an operation to acquire specialist information was not performed, this flow for dietary advice is terminated, and the originating flow is returned to.

Next, display of providable information (refer to S29 in FIG. 2B) will be described using the flowcharts shown in FIG. 7A and FIG. 7B. As was described previously, in step S29 current providable information was displayed in order for the control section 12 implement dietary advice.

If the flow shown in FIG. 7A is commenced, it is first determined whether or not to search for convenience stores or supermarkets (S91). Here, there is a case where the user acquires ingredients and cooks food in accordance with dietary advice of steps S13 and S15. In this case, the control section 12 determines whether or not to search for retail shops such as convenience stores and supermarkets where it is possible to acquire the ingredients, etc., that were shown in steps S13 and S15 (refer to FIG. 2A).

If the result of determination in step S91 is to search for convenience stores and supermarkets, then next items that satisfy conditions are searched for from items on sale, and those stores and product names are displayed (S93). Here, when there are ingredients displayed in step S71, shops such as convenience stores and supermarkets that are selling ingredients that satisfy requirements of step S75, and the names of products at those shops, are displayed on the display section 15.

In order to display shops and products in those shops, it is made possible to access computers and databases constituting a shop and product management system from the information terminal 10. It is assumed that the database etc. obtains information such as cooking methods, etc., that include materials, such as food and desserts, and seasoning. If it is possible to classify not in terms of materials themselves, but into dishes and desserts, etc., with metadata and flags that makes determination possible, what type of health status items are good is available.

It is next determined whether or not a reserving operation has been performed (S95). There will be cases where particular item will be sold out before the user arrives at the store. It is therefore made possible for the user to perform an operation to request that the store reserve that item. In order to do this, enterprises administering the chatbots of the information terminal 10 may build a network for performing reservations with stores such as convenience stores.

If the result of determination in step S95 is that the user has performed a reservation operation, processing is performed in order to pay for and reserve items, and also delivery procedures (if available and desired by the user) are performed, as required (S97). If the user performs electronic settlement, etc., for payments in order to request ingredients at the convenience store in order to implement the dietary advice, reservation procedures are undertaken. It is also possible to complete delivery procedures so as to have the ingredients delivered to the user's location.

Returning to step S91, if the result of determination in this step is that convenience stores and supermarkets will not be searched for, it is next determined whether or not to search for restaurants and eating establishments (S101). This is because there are cases where the user eats out at restaurants, etc. In this case, the control section 12 searches for restaurants and eating establishments where it is possible to eat dishes that satisfy the ingredients and cooking methods that were shown in steps S13 and S15 (refer to FIG. 2A).

If the result of determination in step S101 is to search for restaurants and eating establishments, then next, items that satisfy conditions are searched for from items on sale, and those restaurant and meal names are displayed (S103). Here, for ingredients that were displayed in step S71 and cooking methods that were displayed in step S73, shops such as restaurants that are providing dishes that satisfy the conditions of step S75, and the names of dishes at those stores, are displayed on the display section 15.

It is next determined whether or not a reservation operation has been performed (S105). The information terminal 10 can perform a reservation operation so that the user can eat those dishes at that store (restaurant or eating establishment) at a reserved time in the future. Here, the control section 12 determines whether or not a reservation operation has been performed. In order to do this, enterprises administering the chatbots of the information terminal 10 may build a network for making reservations with stores such as restaurants.

If the result of determination in step S105 is that the reservation operation has been performed, payment and cooking commencement instructions may be issued, and delivery procedures (if available and desired by the user) are followed as required (S107). The user performs payment using electronic settlement, etc., in order to request dishes at the restaurant, etc., in order to implement dietary advice, and requests that cooking be commenced. It is also possible to simply make a reservation. Also, in a case where it is possible for the restaurant, etc., to deliver, a procedure for delivery may be followed. Alternatively, or in addition, a restaurant may make the meal available for pick up by the user.

If the result of determination in step S101 is not to search for restaurants and eating establishments, it is determined whether or not there is in-store mode (S111). There are cases where the user is already within a retail store such as a convenience store, or inside a store that provides dishes, such as a restaurant. Here, the control section 12 may perform determination based on position of the user that has been detected by position detection units, such as GPS, and may perform determination based on a Wi-Fi signal that is provided within the store. In a case where the user is inside the store they may select in-store mode directly.

If the result of determination in step S111 is in-store mode, items that conform to search criteria are searched for and displayed, and a custom (arranged) request is issued (S113). In a case of already being in a retail store, such as a convenience store, or being in a store that provides dishes, such as a restaurant, products (dishes) that specify conditions such as ingredients and cooking methods of steps S71 to S75 are searched for, and displayed on the display section 15. Alternatively, conditions may be customized and requests issued to that store.

If the result of determination in step S111 is not in-store mode, a request or the like is issued to a dedicated manufacturer or commercial kitchen or commercial food distribution facility (S115). Here, the control section 12 issues a request to a specialist manufacturer capable of providing ingredients, or combinations of ingredients and food preparation (e.g., cooking) methods, that satisfy conditions of steps S71 to S75, for example, a manufacturer who performs a delivery service.

If the processing of steps S115, S113 and S107 has been executed, or if the result of determination in step S105 is that a reserve operation has not been performed, or if a reserve operation has not been performed in step S95, or if the processing of S97 has been executed, the flow for providable information display is terminated, and the originating flow is returned to.

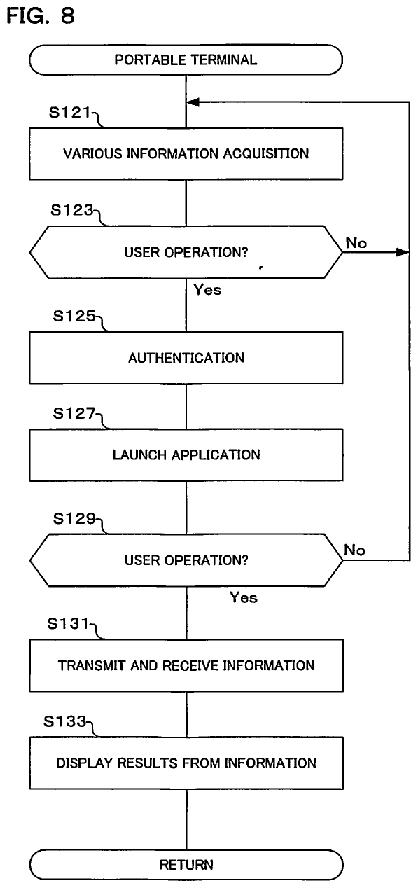

Next, operation of the portable terminal will be described using the flowchart shown in FIG. 8. This flow shows operation in a case where the information terminal 10 functions as a portable terminal. The information terminal 10 may be an appliance other than a smartphone, but in this embodiment description will be given for operation assuming a smartphone. This smartphone will be described as always being turned on.

If the flow shown in FIG. 8 is commenced, first, various information is acquired (S121). Here, the information terminal 10, as the portable terminal, acquires various information. The information terminal 10 normally performs communication with relay stations, and acquires various information, such as mail information, news information, etc.

Once various information has been acquired, it is next determined whether or not there has been a user operation (S123). In a case where the user instructs operations to the portable terminal, the operation section 16 (for example, icons and various operation buttons, etc., on the display section 15) is operated, and so in this step the control section 12 determines whether or not operations by the operation section 16, for example, touch operations on applications for activating the information terminal 10 and icons for activating application software for specified use, have been performed. If the result of this determination is that user operations have not been performed, processing returns to step S121.

If the result of determination in step S123 is that there has been user operation, authentication is performed (S125). Here, the control section 12 detects whether the actual user has performed an operation. For example, it is determined whether or not the actual user has performed an operation using a password method.

If authentication has been performed in step S125, next, an application is launched (S127). Since the person in question has performed an operation, the control section 12 launches application software that has been designated by the user.

Once the application software has been launched, it is next determined whether or not a user operation has been performed (S129). Here, similarly to step S123, it is determined whether or not the user has operated the operation section 16. If the result of this determination is that a user operation has not been performed, processing returns to step S121.

If the result of determination in step S129 is that there was a user operation, transmission and reception of information is performed (S131). In a case where the information terminal 10 is a smartphone transmission and reception of information is performed in order to perform transmission and reception between the mobile phone and the Internet, etc. Also, in a case where there are chatbot functions, information such as the user status information 71 and the diagnosis and examination information 72 is received from medical facilities and examination agencies such as the municipal hospital A 20 and the district hospital B 25 (refer, for example, to S1, S3, and S5 in FIG. 2A).

Once transmission and reception of information has been performed, next, results are displayed based on the information (S133). Here, the control section 12 performs display based on information that was acquired in step S1. For example, normal advice and dietary advice shown in FIG. 2A (S9, S13, S15), and alternative advice and providable information shown in FIG. 2B (S23, S29) may be displayed. If results based on information have been displayed, the flow for the portable terminal is terminated, and the originating flow is returned to.

Next, an example of a database (DB) storing dietary advice will be described using FIG. 9. If an examination or surgery etc. are performed, dietary restrictions are placed on the user before and after the examination or operation. With this embodiment appropriate dietary advice is given in accordance with content of the examination operation (refer, for example, to S9, S13, and S15 in FIG. 2A). Regarding this dietary advice, as well as performing inference using the inference engine 11 within the information terminal 10, a DB for dietary advice may be stored in the storage section 14, with dietary advice being retrieved from the DB in accordance with conditions of the user, and displays to the user.

FIG. 9 is an example of a DB that stores dietary advice in a case of the user having undergone an examination using a stomach endoscope or large intestine endoscope. In a case of having undergone an examination using a stomach or intestine endoscope also, dietary advice after that examination will depend on treatment at the time of examination. With the example shown in FIG. 9, a case where body tissue is collected, and a case where body tissue is not collected, at the time of examination, a case where pathological examination has been performed, and a case where polyps have been excised, are shown.

For example, in a case where large intestine polyps have been excised, at the time of a large intestine endoscopic examination, dietary advice aimed at easily digestible food is displayed for between five and seven days after excision. It is particularly important for the user to pay special attention to diet content from the day of an operation until the following day (e.g., the first 24-48 hours following the procedure). As dietary advice, advice is given to endeavor to create menus and recipes that are good for the body. For example, care should be taken so as to avoid foods such as fatty foods, high fat foods, stimulants such as spices and carbonic acid, and alcohol. On the other hand, as preferred food wheat noodles, chicken and egg rice bowl, ramen, stew, and curry, etc., may be displayed.

As foods that should be avoided, there are, for example, seeded fruits and jams, such as strawberries, kiwi fruit, and watermelon, etc., as well as types of mushrooms, kelp, seaweed, leafy vegetables, burdock, corn, arum root, green soybeans, dark edible seaweed and dried radish strips, buck wheat noodles, and tempura and fried food. On the other hand, as preferred foods there are boiled udon, rice porridge, types of potatoes, bananas, apples, purine, jelly, eggs and egg dishes, sliced fish, candy, and types of soup, etc.

Besides this, as dietary advice at the time of polyp excision, the fact that there is no problem in drinking non-alcohol drinks. Further, display may be performed to encourage always consulting with the physician in charge in a case where anticoagulants and antiplatelet drugs are being taken, or when experiencing abdominal pain, bloody stools or fever, etc., after intestinal polyp excision.

Also, the database shown in FIG. 9 is shown for dietary advice in a case where examination or treatment have been performed using a stomach or large intestine endoscope. However, the examination and treatment are not limited to those using an endoscope, and there may also be a database for dietary advice in the case of other examination appliances and treatments, or other medical procedures, including surgery, dental work, etc. A database may also be prepared for providing dietary advice taking into consideration body condition and lifestyle habits of the user, without being limited to examination and treatment at a medical facility. This database stores advice relating to shops and services that are capable of providing foodstuffs, ingredients, and beverages, etc., that are suitable for improving the user's body condition, and maintaining health, based on user status information. Dietary advice is retrieved from the database on the basis of user condition, and if this advice is provided it is made possible for the user to easily ingest necessary diet items. Also, if active components and drug interactions are also stored in the database information, it is possible to also provide advice even in cases where a plurality of materials are combined.

Next, operation of a CPU of a medical appliance will be described using the flowchart shown in FIG. 10. Various medical appliances, such as a stomach endoscope or large intestine endoscope, etc., are provided at medical facilities and examination agencies such as the municipal hospital A 20 and the district hospital B 25. CPUs within the medical appliances may be provided for individual medical appliances. Alternatively, a medical server provided within medical facilities or examination agencies may be connected to individual medical appliances, and this medical server, etc., may fulfill the functions of CPUS in all medical appliances arranged within the medical facilities.

If the flow for a medical appliance CPU shown in FIG. 10 is commenced, first, patient information is input (S141). Here, the CPU is input with information on a patient who has undergone an examination. For example, information such as name of the patient, registration number of the patient, examination date, examination items, information transmission destination information for the patient (for example, address, etc., of the information terminal 10), etc., may also be input. Further, patient profile, lifestyle habit information, medical history, and medication history, etc., may also be input as patient information. If basic information on the patient is stored in the databases of medical facilities and examination agencies, etc., it is possible to link to this information. The patient information is transmitted to the information terminal 10 in step S149 which will be described later, including diagnosis and examination information 72 (refer, for example, to S3 in FIG. 2A, and S51 in FIG. 4).

Next, measurement setting is performed (S143). Here, the CPU sets content to be measured by each appliance, in accordance with examination items. For example, in the case of a stomach endoscope or large intestine endoscope examination, conditions for storing images from the endoscopic examination are set. Also, if there is an x-ray measurement device, settings that are necessary in order to execute examination, such as x-ray dosage setting and projection position settings, etc., are performed.

Next, examination information is acquired as a result of operation by a health care professional (S145). Here, a health care professional such as a doctor operates the medical appliance, and examination information at this time is acquired. For example, in the case where a doctor operates a stomach endoscope or large intestine endoscope, appropriate images at the required location are acquired, and stored. Data such as endoscopic images that have been acquired here are transmitted to the information terminal 10, and stored in the storage section 14.

It is next determined whether or not examination has been completed (S147). In the event that the health care professional has completed a specified examination, if that fact is input to the medical appliance, determination is performed on the basis of that information. If it is possible to perform automatic determination at the medical appliance side, those determination results are complied with. If the result of this determination is that the examination is not completed processing returns to step S145, and examination continues.

If the result of determination end step S147 is that the examination has been completed, the acquired examination item information and required examination information is transmitted in association with patient information (S149). For example, in the case of a stomach endoscope or large intestinal endoscope examination, information as to whether it is a stomach endoscope examination or is a large intestinal examination, and information as to whether tissue sampling has been performed or whether excision of polyps has been performed, etc., is transmitted to the information terminal 10 of the patient. If these items of information are received by the information terminal 10 (refer, for example, to S131 in FIGS. 8, and S3 in FIG. 2A), it is possible to provide dietary advice to the user on the basis of these items of information (refer, for example, to S9, S13 and S15 in FIG. 2A).

Next, operation for training image data collection will be described using the flowchart shown in FIG. 11. As described previously, the computer 30 has the data collection section 31 and the customized learning section 33, whereby training data for learning is generated by subjecting data that has been collected by the data collection section 31 to annotation, the customized learning section 33 performs learning using the training data, and an inference model is generated. The inference engine 11 within the information terminal 10 infers dietary advice using this inference model, and displays dietary advice based on the inference results. The flowchart shown in FIG. 11 is implemented by a control section (processor) such as a CPU within the computer 30 (or data collection section 31) executing in accordance with programs.

If the flow for collection of training image data shown in FIG. 11 is commenced, first, materials are input (S151). In a case where an examination or operation has been undertaken at a medical facility or examination agency, they will be dietary restrictions and recommended diets before and after the examination or operation. Materials for these dietary restrictions on recommended diet (ingredients and seasonings, etc.) are input in the form of text data, etc. This input may be by manual operation, or may be automatically input by the data collection section 31 based on data that has been contributed on the Internet, or care food data sets such as shown in FIG. 1C.

Once the materials have been input, next, food images are retrieved (S153). The data collection section 31 collects food images that include materials that have been input in step S151. For example, images that contain ingredients that were input in step S151 are collected from among images of food that has been provided at the large hospital 51 or nursing facility 53 (refer, for example, to images 1 to 3 of the care food sets 77a to 77c). If there is a large hospital (including a place of treatment, or long term hospital where the user lives) or nursing facility, there is information on what type of symptoms there are, and what type of food menus are effective, for every age and gender. If there is hospital food and care food supply to hospitals, etc., there will be a lot of material that can be used as training data at the time of providing dietary advice. Materials and cooking methods for these food menus are collected and made into training data. Photographs of cooking site information 76b that have been uploaded onto the Internet (for example, Cookpad (registered trademark), etc.) may also be collected.

If food images are retrieved, then next, a circle (◯) is attached to food that only has good ingredients (given information about the user), and a cross (X) is attached to food that includes detrimental ingredients (S155). For images that have been retrieved in step S153, the data collection section 31 assigns a circle (◯) for food that has been cooked with only ingredients that it is good for the user to eat. On the other hand, a cross (X) is assigned to images for foods that includes ingredients that would be detrimental if eaten by the user. It should be noted that whether ingredients are good for the user to eat or would be detrimental for the user to eat will differ depending on the examination, etc., that the user has undergone, will differ depending on the number of days since the examination, and/or user information such as age, sex, height, weight, preexisting medical conditions, etc. The ◯ and X marks are attached taking this into account. Depending on the ingredients, there may be some things that are more highly recommended than simply being good, and in this case a double circle (⊚) may be attached. Also, a triangle (Δ) may be attached to ingredients that can tentatively be eaten. Levels of evaluation are not limited to two levels or four levels, and may be appropriately changed, and evaluation levels may also be shown as numerical values, and with some other visual based indicator. There are cases where a plurality of dishes exist within an image, and in that case a ◯ or X is assigned for each dish. The evaluation symbols such as ◯ and X can also be variously changed.

If annotation has been attached to images in step S155, it is next determined whether or not a specified number of images have been acquired (S157). In generating an inference model of high reliability by machine learning, a lot of training data are required. Here, the data collection section 31 acquires a specified number of images that are required to perform machine learning, and determines whether it was possible to assign ◯ or X to these images. If the result of this determination is that a specified number of images have not been acquired, processing returns to step S51.

On the other hand, if the result of determination in step S157 is that a specified number of images have been acquired, next, ◯ and X are assigned to the images as annotation, the images are made into training data, and machine learning is executed (S159). Here, the data collection section 31 performs annotation of ◯ or X to food images in accordance with determination result in step S155, and generates training data. Once training data has been generated, the customized learning section 33 generates an inference model using the training data.

Once machine learning has been performed, it is next determined whether or not reliability of the inference model that has been generated is OK (S161). Here, the customized learning section 33 performs determination based on whether or not reliability of the inference model data has been generated is greater than or equal to a predetermined value. Reliability is obtained from a proportion of images that match a correct solution at the time images for which a predetermined correct solution is known are input to the inference model.

If the result of determination in step S161 is that reliability is not OK, optional selection of training data is performed (S165). If reliability is low, there will be cases where a training data population is inappropriate. The data collection section 31 therefore performs optional selection of training data in order to improve reliability. If optional selection of training data has been performed, processing returns to step S159 and machine learning is executed again.

On the other hand, if the result of determination in step S161 is that reliability is OK, the inference model is transmitted (S163). Here, the communication section (communication circuit) within the computer 30 transmits the inference model that has been generated by a customized learning section to the communication section 13 within the information terminal 10. If the inference model has been received by the information terminal 10, it is set in the inference engine 11. The inference engine 11 provides dietary advice using the inference model data has been set (refer, for example, to S13 and S15 in FIG. 2A). If the inference model has been transmitted, the flow for collection of training image data is terminated, and the originating flow is returned to. Note that a user may take images of a dish or food before and after they are done eating. If there is food remaining after the user finishes eating, the quantity of food eaten can be estimated from a comparison of the before and after images.

If images are inferred using this type of inference model, it is possible to provide dietary advice in accordance with dietary restriction of the user for every dish (refer, for example, to S13 and S15 in FIG. 2A). Appearance of this dietary advice is shown in FIG. 12. In FIG. 12, if the user points the information terminal 10 at set dishes made up of three items, the set dishes are displayed on the display section 15. At this time, for each dish, ◯ is displayed on dishes that have been prepared using only ingredients that it is good user to eat, and X is displayed on dishes that include detrimental ingredients. As a result, there is the advantage that it is possible to easily know whether food is good or bad to eat, simply by the user pointing the information terminal 10 at dishes and acquiring images of the dishes. Although the foregoing example concerned a photographed image, inference using images of meals retrieved from internet may be performed instead, or in addition. . . . The information used and/or provided by a POS system includes image information of product (ingredient, food and drink, etc.) according to trade name. Although this example for making an inference model uses training data based on images, this is not limiting and the inference model may be made using information instead or, or in addition to, images of cooked information. For example, it may also be possible to make the inference model from text information about how the meal was prepared (e.g., cooked), text information about the name of the meal, text information about the ingredients, etc. . . . That is, text information is annotated by whether the state of the user and/or patient is good or bad. This obtained training data may then be used to make the inference model. Further, such an inference model may be used in every embodiment.

Also, as shown in FIG. 13, it is difficult for a user who is being subjected to dietary restriction after having undergone an endoscopic examination, or a user who as allergies and is being subjected to dietary restriction, to search for and purchase a lunch box that meets those dietary restrictions at a convenience store. In this case, it is possible to easily determine what can be purchased by pointing the imaging section of the information terminal 10 at food in the lunchbox, as was shown in FIG. 12. Even in a case where there are many lunchboxes and there are many types of ingredients subjected to dietary restriction, there are cases where, among the lunchboxes on sale over the counter, there are none that satisfy requirements. In this type of situation, it is also possible to order lunchboxes that are arranged for the intention of a user who is subject to dietary restriction by means of the information terminal 10, at a convenience store, or to arrange delivery of other dishes, as shown in FIG. 13.

Also, even if the user themselves performs ordering, in cases such as where permission is allowed contractually, etc., it is made possible for a control section of a shop or service system (for example a store server) to acquire user information such as described above, and it is also possible to have a scheme where a store, etc., prepares dishes and ingredients suitable for the user in advance. It is possible to configure a system whereby user status is determined, ingredients that are suitable for that status, and meals that use those ingredients, and cooking methods, are inferred, and this information is provided to the user. Information for procuring corresponding ingredients is output, information on those ingredients and food preparation (e.g., cooking) methods is output to an associated factory, and products that meet those specifications can be delivered by a specified time. This specified time must be before a time when the user will want to purchase those products. A time when the user will want to purchase things may be a time until passing or arriving at that shop, or may be at the time of breakfast, lunch, or evening meal, or correspond to another time. Being able to provide such products is preferable in making it possible to be able to supply information to the user. Provision of this type of information may also be performed in previously described steps S27 to S33 (refer to FIG. 2B).

A store server that performs services as described above may be a product management server that manages product availability, at the time of accounting with a cash register or the like, or at the time of storing products. The store server shall associate information for health benefits of types of diet, types of snacks, and types of drinks that are handled, with that inventory information. Alternatively, an arrangement where it is possible to search for these items of information from product names may be also provided. A store server, in cooperation with a wireless communication section, is capable of outputting information as to what product lists and recommended products can be displayed on a user terminal. Recommendation information that has been customized to a corresponding user (things conforming to health conditions of that user) may be distributable by wireless. Besides this, information may be shared by cooperation with other stores and store servers or the like by means of the Internet or dedicated lines, and product management may be streamlined. Using this system, it is possible to store necessary products for a required period, and to advertise and sell these products.

Systems and methods providing such functions are called POS systems and POS methods. This is an abbreviation for “point of Sale”, and these are systems and methods for collating and analyzing daily sales for each product type, and using this data in management. A computer that controls a POS system can handle input information from a reader (scanner) such as a bar code. If the computer of a POS system reads in a bar code using a scanner of a cash register or the like at the time of payment or receipt, for example, it is possible to register, collate and arrange products that have been received or shipped, and the number of such products, and to store in a storage section for product management. At the time this cash register information is input, it is possible to handle together with customer profile information, etc.

A POS computer is also capable of being made into a large system by being connected to a network. Data that has been collated is also shared with headquarters, and it becomes possible to perform inventory control and sales analysis, and stock taking of top-selling products, using information that can be collated by a computer at the headquarters. Naturally, position information and opening times of each store can also be managed. Accordingly, what type of products are at which stores, etc., can be easily searched for and confirmed if there is cooperation with information that has been stored in storage sections of computers at headquarters. For example, by comparing stores that are close to a hospital with stores in other regions, in accordance with health condition and diagnosis results, it is possible to ascertain information for each store, such as products that are suitable to classes of user that sell a lot, and it is possible to provide selection of goods that are appropriate to that class of user. However, if patients who have gone out of that hospital hope to be able to purchase similar products close to their own residence, there is a possibility that this will not always be possible. As shown in this embodiment, if there is a service that acts in cooperation with the POS system, it is possible to immediately confirm which store should be visited to be able purchase expected items etc. Also, it is possible for stores where items are not ready to order in those products by cooperating with stores where the items are ready, and a POS system.

The above described bar codes for identifying products are capable of showing information such as international common commodity code, product code, product name, prices, etc. Since it is easy to ascertain information on what store products have been sold at, how many of the products are in stock, what is the most popular product, etc., many stores have adopted the POS system. If there is food and drink etc., materials (for example, ingredients) and food preparation (e.g., cooking) methods, etc., are stored in association in product information that is being managed by the POS system in association with product names, and it is possible to determine at what store it will be possible for a user to obtain meals that are suitable to their own body condition by simply making these items of information searchable, as shown in this embodiment. Here also, if materials (such as ingredients) and food preparation (e.g., cooking) methods for products are made into a database, it is possible to be able to determine easily that products that a user is not aware of will actually be a problem for that user.

In the description above, specific symptoms being prominent sections, data, affected parts being approached directly for the purpose of treating a specified location, and treatment being provided by removing causes using methods such as medication and operations, have been described solely on the assumption of so-called western medicine. Although it has not been mentioned positively thus far, in order to treat physical constitution from the root, it is also possible to apply this embodiment to Oriental medicine or Chinese medicine, as medical science that also includes acupuncture and moxibustion, and diet cures, and it goes without saying that there is high compatibility with this embodiment.

With Oriental medicine, on the basis of a way of thinking that “a person's body is perceived as part of nature”, there are such features that entire body balance is comprehensively reconsidered. Accordingly, with Oriental medicine, an approach is taken of reconsidering the situation from body composition and lifestyle habits, etc., with the view that handling this will lead to good health. It is therefore known that Oriental medicine is also effective in disorders with no name and when a patient is not yet ill, etc. Also, since herbal medicine that includes active components is used in treatment, it is possible to expect that good health will continue by appropriately following a diet that has the same components.

The “body composition and lifestyle habits” mentioned here is often something that the user in question is not aware of, and there are cases where it is possible to analyze this objectively from inquiries and storage of lifestyle habits. For example, with Chinese medicine there is classification into “seeing” such as complexion and expression, attitude and physique, etc., hearing, such as listening to voice volume and tone, how the subject speaks, how they cough, appearance of phlegm (how they clear their throat), sound of breathing, etc., asking, where the subject is asked about subjective symptoms, illnesses they have suffered from up to now, preferred foods, and lifestyle, etc., and touching the body to confirm the condition of that body. There is also similar examination in western medicine.

Since it is possible to acquire various information (status information of the user) using chatbot functions secured by means of a portable terminal or information terminal, etc., and sensors of those terminals, giving advice taking into consideration the previously described body condition and lifestyle habits may also be included in this embodiment. Specifically, body composition of the user is classified from status information of the user (for example, with Chinese medicine body composition is classified into “proof”, and “air, blood, and water”), and there are cases where body composition of the user can be ascertained based on this classification. It is therefore possible to search a database in which recommended diets, etc., are arranged in accordance with user type, and to provide advice relating to shops that are capable of providing foodstuffs, ingredients and drinks, etc., that are suitable for improvement of body composition and continued health, or to provide advice relating to services. If these items of advice are provided, it is possible for the user to easily be able to ingest necessary meals. Depending on the shop, salespeople may also be consulted. Also, if active ingredients and drug interactions, etc., are stored in database information that contains combinations of these foodstuffs, ingredients, drinks, etc., and ingredients and food preparation (e.g., cooking) methods, advice also becomes possible by checking on the plurality of materials constituting dishes. If this type of advice is possible, it becomes possible to only ingest effective things, and to ingest meals while avoiding things that lose effect.

In this way, according to this embodiment, it is possible to perform centralized control of advising the user about things that are permitted to be ingested or beneficial, and it is possible to monitor patient behavior outside of the hospital. Further, if drugs that have being prescribed are also stored in a database, it becomes possible to ascertain relationships between prescribed drugs and diet, etc. Specifically, it is possible for medical facilities such as hospitals and clinics to monitor the health of the user while cooperating with various shops and services, or by cooperation between shops, and between service.

Also, if the information terminal 10 is a portable terminal that has an image input section for inputting images of ingredients that have been laid out in a shop or on a table, or meals that have been prepared, then, as was described previously, it is possible to output advice for displaying suitability for ingestion by the user, for every position of individual items within an image (refer to FIG. 12). At the same time as performing this display, that information is shared with cooperating facilities and specialists, and it is also possible to receive advice for ingredients and drinks that should be ingested from the cooperating facilities that have shared the information.

Since taking pictures of food is performed before meals, medicine taken before meals may be instructed to the user using this timing. At an appropriate time after eating, it is possible to simply output advice to avoid forgetting to drink and misuse, by instructing the user about medicines to be taken after eating, using voice, or superimposed display or icon display for separate screens.

As has been described above, with one embodiment of the present invention, diagnosis and examination information of the user is input (refer, for example, to the control section 12 and communication section 13 in FIG. 1B, and to S3 in FIG. 2A), user status information is input (refer, for example to the control section 12 and communication section 13 in FIG. 1B, and to S5 in FIG. 2A), and advice relating to diet is output to the user based on the diagnosis and examination information and status information (refer, for example, to the control section 12 in FIG. 1B, and to S13 and S15 in FIG. 2A). It should be noted that status information of the user may be input without inputting diagnosis and examination information of the user, and advice relating to diet may be output based on this status information. Also, when outputting advice relating to diets, subjective symptoms and health information of the person in question may be used instead of, or in addition to, the diagnosis and examination information. In this way, according to this embodiment it is possible to give advice relating to diet to the user based on condition of the user, and diagnosis on examination results. Specifically, it is possible to give advice relating to appropriate diet in accordance with user diagnosis and examination results, and status, without being limited to when the user has been admitted to hospital.

Also, with one embodiment of the present invention, status information of the user is input (for example, S5 in FIG. 2A), and advice relating to shops or services that are capable of providing meals suitable for the user are output in accordance with this status information (refer, for example, to S29 in FIG. 2A, and to FIG. 7A and FIG. 7B). As a result, it is possible to give advice relating to appropriate diet in accordance with user condition.

Also, with one embodiment of the present invention images of ingredients or dishes that have been prepared are input (for example, S77 in FIG. 5), and advice for displaying suitability for the user to consume is output for these images that have been input (refer, for example, to S79 in FIG. 5, and to FIG. 12). As a result, it is possible for a user to easily know whether or not it is suitable for the user to eat something.

It should be noted that with the one embodiment of the present invention, description has mainly been given using an example of a smartphone as an information terminal. However, the information terminal 10 is not limited to a smartphone, and may also be an information device such as a smart home appliance (including an AI speaker), digital home appliance, or personal computer, etc. If it is possible to connect the information terminal to a communication network, such as the Internet, communication is performed with various devices, diagnosis and examination information and user status information is collected, and it is possible to give advice relating to diet based on these items of information.

Also, with the one embodiment of the present invention, although description has been given of examples of determination of a logic base that uses a database, etc., or determination by inference using machine learning, either of these determinations may be used in this embodiment. With a database also, there are also cases of use at the time of creating an inference model (at the time of learning) by making this database into training data, and this is also one example of determination using a database. Also, in the determination process hybrid type determination may also be performed partially using respective merits. For example, there may be a configuration where some determinations of food names from food images use an inference model, while materials and cooking methods for those foods are determined using database retrieval, and it is made possible to retrieve what effect materials, etc., that have been determined have on the body using a database, but in a case where material names are ambiguous inference may be used, while in rigorous cases database determination may be used.

Also, with the one embodiment of the present invention, the control section 12 has been described as an IT device comprising a CPU, memory, and HDD, etc. However, besides being constructed in the form of software using a CPU and programs, some or all of these sections may be constructed with hardware circuits, or may have a hardware structure such as gate circuitry generated based on a programming language described using Verilog, or may use a hardware structure that uses software, such as a DSP (digital signal processor). Suitable combinations of these approaches may also be used. Also, without limiting to a CPU, there may be components that fulfill functions as a controller, or processing for each of the above described section may be performed by one or more processors constructed as hardware. For example, each section may be a processor constructed as respective electronic circuits, and may be respective circuits sections of a processor that is constructed with an integrated circuit such as an FPGA (Field Programmable Gate Array). Alternatively, a processor that is constructed with one or more CPUs may execute functions of each section, by reading out and executing computer programs that have been stored in a storage medium.

Also, in the one embodiment of the present invention, it has been described that the information terminal 10 comprises an inference engine 11, control section 12, communication section 13, storage section 14, display section 15, and operation section 16. However, these sections do not need to be provided within a single unit, and if there is connection by means of a communication network such as the Internet, for example, it is possible to have a configuration where each of the above sections is dispersed.

Although some example embodiments considered dietary needs of a particular person, some other embodiments might consider medical conditions of more than one person, such as husband and wife for example. For example, a husband might have medical condition A, and his wife might have medical conditions B and C. In this scenario, any shared meal (that is, a meal to be eaten by both the husband and the wife) should meet requirements suitable to conditions A+B+C.

Also, among the technology that has been described in this specification, with respect to control that has been described mainly using flowcharts, there are many instances where setting is possible using programs, and such programs may be held in a storage medium (such as a non-transitory storage medium) or storage section. The manner of storing the programs in the storage medium or storage section may be to store them at the time of manufacture, or by using a distributed storage medium, or they be downloaded via the Internet.

Also, with the one embodiment of the present invention, operation of this embodiment was described using flowcharts, but procedures and order may be changed, some steps may be omitted, steps may be added, and further the specific processing content within each step may be altered. It is also possible to suitably combine structural elements from different embodiments.

Also, regarding the operation flow in the patent claims, the specification and the drawings, for the sake of convenience description has been given using words representing sequence, such as “first” and “next”, but at places where it is not particularly described, this does not mean that implementation must be in this order.

As understood by those having ordinary skill in the art, as used in this application, ‘section,’ ‘unit,’ ‘component,’ ‘element,’ ‘module,’ ‘device,’ ‘member,’ ‘mechanism,’ ‘apparatus,’ ‘machine,’ or ‘system’ may be implemented as circuitry, such as integrated circuits, application specific circuits (“ASICs”), field programmable logic arrays (“FPLAs”), etc., and/or software implemented on a processor, such as a microprocessor.

The present invention is not limited to these embodiments, and structural elements may be modified in actual implementation within the scope of the gist of the embodiments. It is also possible to form various inventions by suitably combining the plurality structural elements disclosed in the above described embodiments. For example, it is possible to omit some of the structural elements shown in the embodiments. It is also possible to suitably combine structural elements from different embodiments.

What is claimed is:

1. A method for issuing dietary advice to a user, the method comprising:
acquiring time information indicating at least one of a first date or a first time when the user underwent a medical examination, medical diagnosis or medical procedure at a medical institution;
acquiring a first image including meals taken at a second date and time;
calculating an elapsed time based on the time information and the second date and time;
accessing a database containing care food information including care food images organized by both (i) a type of medical examination, medical diagnosis, or medical treatment and (ii) the elapsed time;
determining the care food information based on (i) the medical examination, medical diagnosis or medical procedure at the medical institution and (ii) the elapsed time calculated;
determining a suitability of a dish or at least one food ingredient in the first image according to the at least one of the care food information; and
responsive to determining the suitability, generating a second image by superimposing information indicating the suitability on the first image.

2. The method according to claim 1, further comprising:
controlling a display device to display the second image subsequent to generating the second image.

3. The method according to claim 1, wherein
the suitability is determined for an individual dish in the first image.

4. The method according to claim 1, wherein
the suitability is determined for each of the at least one ingredient in the first image, and/or for an individual dish in the first image.

5. The method according to claim 4, further comprising
displaying information of the suitability superimposed on the first image according to the position of each of the at least one ingredient and/or the position of the individual dish as the dietary advice regarding the user's diet.

6. The method according to claim 1, further comprising
determining a recovery time constant according to a standard number of days required for recovery based on the required medical examination, medical diagnosis, or medical treatment information; and
comparing the elapsed time calculated and the standard number of days required for recovery based on the required medical examination,
wherein the suitability is determined responsive to comparing the elapsed time calculated and the standard number of days.

7. The method according to claim 1, wherein
the suitability is determined responsive to comparing the elapsed time calculated and a threshold, and
wherein the threshold is a standard number of days required for recovery based on the required medical examination, medical diagnosis, or medical treatment information.

8. The method according to claim 7, further comprising:
determining a recovery time constant according to the standard number of days;
determining whether the recovery time constant is longer than the standard number of days; and
responsive to a determination that the recovery time constant is longer than the standard number of days, adding a number of days to the threshold.

9. The method according to claim 1, further comprising:
obtaining an endoscopic image of the user;
determining whether there is a polyp in the endoscopic image; and
responsive to determining that there is a polyp in the endoscopic image, (i) changing the dietary advice based on a size and/or a position of the polyp, and (ii) changing a period for determining a suitability of the dish or the at least one food ingredient in the first image when giving the dietary advice.

10. The method according to claim 1, further comprising:
obtaining an endoscopic image of the user;
determining whether there is a wound image from a procedure in the endoscopic image;
responsive to determining that there is the wound image from the procedure in the endoscopic image, (i) determining a period until recovery, and (ii) changing, using a period until recovery, a suitability of the dish or the at least one food ingredient in the first image when for giving the dietary advice.

11. A control device for issuing dietary advice to a user comprising:
one or more processor configured to:
acquire time information indicating at least one of a first date or a first time when the user underwent a medical examination, medical diagnosis or medical procedure at a medical institution;
acquire a first image including meals taken at a second date and time;
calculate an elapsed time based on the time information and the second date and time;
access a database containing care food information including care food images organized by both (i) a type of medical examination, medical diagnosis, or medical treatment and (ii) the elapsed time calculated;
determine at least one of the care food information based on (i) the medical examination, medical diagnosis or medical procedure at the medical institution and (ii) the elapsed time calculated;
determine a suitability of a dish or at least one food ingredient in the first image according to the at least one of the care food information; and
responsive to determining the suitability, generating a second image by superimposing information indicating the suitability on the first image.

12. The control device according to claim 11, wherein the one or more processors being configured to further:
control a display to display the second image subsequent to generating the second image.

13. The control device according to claim 11, wherein
the suitability is determined for the dish in the first image.

14. The control device according to claim 11, wherein
the suitability is determined for each individual one of the at least one ingredient in the first image.

15. The control device according to claim 11, wherein
the suitability is determined for the dish in the first image, and/or for each individual one of the at least one ingredient in the first image, and wherein
the one or more processors being configured to control a display to display information of the suitability superimposed on the first image according to the position of the dish and/or the position of each individual one of the at least one ingredient as the advice regarding the user's diet.

16. The control device according to claim 11, wherein
the one or more processors are configured to:
determine a recovery time constant according to a standard number of days required for recovery based on the required medical examination, medical diagnosis, or medical treatment information; and
compare the elapsed time calculated and the standard number of days required for recovery based on the required medical examination,
wherein the suitability is determined responsive to comparing the elapsed time calculated and the standard number of days.

17. The control device according to claim 11, wherein
the suitability is determined responsive to comparing the calculated elapsed time and a threshold, and
the threshold is a standard number of days required for recovery based on the required medical examination, medical diagnosis, or medical treatment information.

18. The control device according to claim 17, wherein
the one or more processors are configured to:
determine a recovery time constant according to the standard number of days;
determine whether the recovery time constant is longer than the standard number of days; and
responsive to a determination that the recovery time constant is longer than the standard number of days, adding a number of days to the threshold.

19. The control device according to claim 11, wherein
the one or more processors are configured to:
obtain an endoscopic image of the user;
determine whether there is a polyp in the endoscopic image; and
responsive to determining that there is a polyp in the endoscopic image, (i) change the dietary advice based on a size and/or a position of the polyp, and (ii) change a period for determining a suitability of the dish or the at least one food ingredient in the first image when giving the dietary advice.

20. The control device according to claim 11, wherein
the one or more processors are configured to:
obtain an endoscopic image of the user;
determine whether there is a wound image from a procedure in the endoscopic image;
responsive to determining that there is the wound image from the procedure in the endoscopic image, (i) determine a period until recovery, and (ii) change, using the period until recovery, a suitability of the dish or the at least one food ingredient in the first image when giving the dietary advice.
